(19) United States
(12) Patent Application Publication
(10) Pub. No.: US 2024/0245730 A1
(43) Pub. Date: Jul. 25, 2024

(54) SYNTHETIC PROTEIN FOR INDUCING IMMUNE TOLERANCE

(71) Applicant: Wisconsin Alumni Research Foundation, Madison, WI (US)

(72) Inventors: Jacques GALIPEAU, Madison, WI (US); Pradyut PAUL, Madison, WI (US)

(21) Appl. No.: 18/561,849

(22) PCT Filed: May 17, 2022
(86) PCT No.: PCT/US2022/029653

§ 371 Date: Nov. 17, 2023

Related U.S. Application Data
(60) Provisional application No. 63/189,359, filed on May 17, 2021.

(51) Int. Cl. A61K 35/39; A61P 3/10; A61P 37/06; C07K 14/705; C12N 5/071; C12N 9/02; C12N 15/86

(52) U.S. Cl. CPC A61K 35/39; A61P 3/10; A61P 37/06; C07K 14/70532; C12N 5/0676; C12N 9/0069; C12N 15/86; C07K 2319/02; C07K 2319/03; C12N 2510/00; C12N 2740/15043; C12Y 113/11052

(57) ABSTRACT

The present invention provides fusion proteins that induce local immune tolerance. The fusion proteins comprise peptides derived the immunoregulatory proteins programmed death ligand-1 (PD-L1) and indolamine 2,3-dioxygenase (IDO). Also provided are nucleic acid constructs encoding said fusion proteins, cells comprising said nucleic acid constructs, and methods of transplanting said cells into a subject.

### CROSS-REFERENCE TO RELATED APPLICATIONS

This application claims priority to U.S. Provisional Application No. 63/189,359 filed on May 17, 2021, the contents of which are incorporated by reference in their entireties.

### SEQUENCE LISTING

A Sequence Listing accompanies this application and is submitted as an ASCII text file of the sequence listing named “960296_04290_ST25.txt” which is 114,175 bytes in size and was created on Apr. 29, 2022. The sequence listing is electronically submitted via EFS-Web with the application and is incorporated herein by reference in its entirety.

### BACKGROUND

Transplant rejection occurs when the recipient's immune system attacks the donated graft and begins destroying the transplanted tissue or organ. Currently, chronic systemic immunosuppression is the only clinical strategy available to prevent the rejection of allogenic transplants (1). Despite significant improvements in post-transplant immunosuppressive therapies, long-term inhibition of the host immune response still causes serious adverse effects such as opportunistic infections, cardiac and renal toxicity, and increased risk of malignancies (2). Both these adverse effects and the severe shortage of cadaver-derived cells and tissues are major obstacles preventing the broad adaptation of allogenic transplant therapies as treatments for several end-stage human diseases (1, 3-5). For example, islet transplantation is a promising therapy for treatment of type-1 diabetes (T1D)(6-8). But, unfortunately, the majority of islet allograft recipients lose graft function and insulin independence within 3-5 years post-transplant (9). Further, an immunosuppressive regimen that prevents the rejection of xenogeneic transplants has never been established. Thus, there remains a critical and unmet need for a safer and more effective means of inducing immune tolerance to allogeneic or xenogeneic grafts.

### SUMMARY

The present invention provides engineered fusion polypeptides that are based on the inventor's fusion protein, referred to herein as PIDO. The fusion proteins comprise from N-terminus to C-terminus: (a) a PD-L1 peptide comprising at least a portion of the extracellular domain of a PD-L1 protein, (b) a transmembrane domain, and (c) an IDO peptide comprising at least a portion of an IDO protein. In some embodiments, the PD-L1 peptide is capable of binding to PD-1 and the IDO peptide is catalytically active.

In a second aspect, the present invention provides nucleic acid constructs comprising a polynucleotide encoding the fusion proteins described herein operably linked to a promoter.

In a third aspect, the present invention provides cells comprising the nucleic acid construct described herein. Under suitable conditions, the cells express the fusion proteins described herein.

In a fourth aspect, the present invention provides methods of transplanting the cell described herein into a subject.

### BRIEF DESCRIPTION OF THE DRAWINGS

FIG. 1 demonstrates that the PIDO fusion protein is expressed in transduced cells. (A) Schematic depiction of the experiment. Lentivirus was used to transduce pancreatic islets for PIDO expression. (B) Schematic of the PIDO expression construct (top) and the PIDO protein sequence (SEQ ID NO:1; bottom). (C) Predicted 3D structure of PIDO. (D) Lentivirus transduction efficiency in A375 human melanoma cells, detected as expression of the indicated fluorescent reporters. DNA was counterstained with DAPI (blue). C57BL/6 mouse islets were transduced by lentiviruses expressing PD-L1, IDO, or PIDO followed by enzymatic dispersion. The transduced cells were analyzed by (E) flow cytometry (i.e., to measure extracellular PD-L1 expression) and (F) western blot (representative of 3) of extracts from PIDO-expressing mouse or pig islets using an anti-IDO antibody (i.e., to measure intracellular IDO expression). (G) Schematic of the predicted subcellular localization of the PIDO constituent proteins. PD-L1 is displayed on the cell membrane while IDO is tethered to cytoplasmic tail of PD-L1 in the cytoplasm. (H) Kynurenine ELISA to detect IDO catalytic activity (n=4). (I) Mouse islets transduced with constructs for the expression of PD-L1, IDO, or PIDO were compared to unmodified islets in a glucose-stimulated insulin secretion assay after 48 hours of in vitro culture. These results show the insulation secretion at low (2.8G) and high (16.7G) glucose concentration. Data are presented as mean±SEM. (*P<0.05, **P<0.01, ***P<0.001).

FIG. 2 demonstrates that PIDO-expressing allogeneic islets reverse pre-existing chemically induced diabetes in mice. (A) Schematic depiction of the experiment. Diabetes was included with streptozotocin (STZ) and PIDO-expressing allogeneic C57BL/6 mouse islets were transplanted into BALB/c mice. (B) Representative sections of transplanted islet allografts under the kidney capsule (bright field, left, 4× magnification) were stained for insulin (green) and actin (red). DNA was counterstained with DAPI (blue). The original magnification was 20×. (C) Blood glucose measurements taken before and after STZ treatment and after transplantation with engineered allogeneic islets. Five groups were studied: (1) non-diabetic mice without a transplant (“No STZ/Txp”; no STZ, no transplant;n=3; dotted line), (2) diabetic mice transplanted with control islets (“Islets^{Ctrl}”; +STZ, EGFP-expressing transplant; 400 islets; n=4; red), (3) diabetic mice transplanted with PD-L1-expressing islets (“Islets^{PD-L1}”; +STZ, PD-L1-expressing transplant; 400 islets; n=5; diamond symbols, broken line, (4) diabetic mice transplanted with IDO-expressing islets (“Islets^{IDO}”; +STZ, IDO-expressing transplant; 400 islets; n=5; hexagon symbols, broken line), and (5) diabetic mice transplanted with islets that co-express PD-L1 and IDO individually (“Islets^{PL-D1+IDO}”; +STZ, PIDO-expressing transplant; 400 islets; n=5; blue). (D) Blood glucose measurements taken before and after STZ treatment and after transplantation with engineered allogeneic islets, in both the fasted (right) and random-fed (left) state. Three groups were studied: (1) mice without a transplant (“No Txp”; no STZ, no transplant; n=3; dotted line), (2) diabetic mice transplanted with control islets (“Islets^{Ctrl}”; +STZ, EGFP-expressing transplant; 400 islets; n=4; red), and (3) diabetic mice transplanted with PIDO-expressing islets (“Islets^{PIDO}”; +STZ, PIDO-expressing transplant; 400 islets; n=5; blue). (E) Glucose tolerance test (GTT) performed 2 weeks and 10 weeks after transplant. Three groups were studied: (1) mice without a transplant (“No Txp”; no STZ, no transplant;n=3; 2 Wk and 10 Wk, black), (2) diabetic mice transplanted with control islets (“Islets^{Ctrl}”; +STZ, EGFP-expressing transplant; n=5; 2 Wk and 10 Wk, red), and (3) diabetic mice transplanted with PIDO-expressing islets (“Islets^{PIDO+}”; +STZ, PIDO-expressing transplant;n=5; 2 Wk and 10 Wk, blue). Lower panel: area under the curve (AUC) quantification of GTT data. (F) In vivo glucose-stimulated insulin secretion (GSIS) assay performed 2 and 10 weeks after transplantation. Three groups were studied: (1) mice without a transplant (“No Txp”; no STZ, no transplant; n=3; 2 Wk and 10 Wk, black), (2) diabetic mice transplanted with control islets (“Islets^{Ctrl}”; +STZ, EGFP-expressing transplant; n=4; 2 Wk and 10 Wk, red), and (3) diabetic mice transplanted with PIDO-expressing islets “Islets^{PIDO+}”; +STZ, PIDO-expressing transplant; n=5; 2 Wk and 10 Wk, blue). Data are presented as mean±SEM. (*P<0.05, **P<0.01, ***P<0.001).

FIG. 3 demonstrates that PIDO-expressing islet allografts improve hyperglycemia in diabetic NOD mice. (A) Schematic depiction of the experiment. PIDO-expressing allogeneic C57BL/6 mouse islets were transplanted into diabetic NOD mice. (B) Fed blood glucose measurements in NOD mice after transplantation with naïve or PIDO-expressing allogeneic islets. Three groups were studied: (1) normoglycemic mice without a transplant (“Non-diabetic/No Txp”; n=4; black), (2) diabetic mice transplanted with control islets (“Islets^{Ctrl}”; EGFP-expressing transplant; 400 islets; n=4; red), and (3) diabetic mice transplanted with PIDO-expressing islets (“Islets^{PIDO+}”; PIDO-expressing transplant; 400 islets; n=5; blue). Animals that died of diabetes complications (hypoinsulinemia) or that had relapsing diabetes were removed from the analysis at the observed time of death/relapse and are marked on the plot with an * and §, respectively. (C) Stairstep graph showing diabetes relapse incidence in PIDO⁺ allogeneic islet-transplanted and naïve allogeneic islet-transplanted NOD mice. Diabetes relapse (blood glucose>250 mg/dL) was used as the terminal event.

FIG. 4 demonstrates that PIDO does not confer acquired immune tolerance against naïve allogeneic islets. (A) Schematic of the experiment. Diabetes was induced with streptozotocin (STZ) and PIDO-expressing allogeneic C57BL/6 mouse islets were transplanted into BALB/c mouse recipients. The recipients were then rechallenged with STZ or nephrectomy and a second subrenal transplantation in the contralateral kidney was performed. (B) Blood glucose measurements taken before and after STZ treatment, after transplantation with allogeneic islets, after rechallenge with STZ, and after the second transplantation with naïve allogeneic islets. Three groups were studied: (1) mice without a transplant (“No Txp”; no STZ, no transplant; n=3; dotted line), (2) diabetic mice transplanted with control islets (“Islets^{Ctrl}”; +STZ, EGFP-expressing transplant; 400 islets;n=4; red), and (3) diabetic mice transplanted with PIDO-expressing islets (“Islets^{PIDO+}”; +STZ, PIDO-expressing transplant; 400 islets; n=5; blue). (C) Blood glucose measurements taken before and after STZ treatment, after transplantation with engineered allogeneic islets, after rechallenge via nephrectomy, and after the second transplantation with naïve allogeneic islets. Three groups were studied: (1) mice without a transplant (“No Txp”; no STZ, no transplant; n=3; dotted line), (2) diabetic mice transplanted with control islets (“Islets^{Ctrl}”; +STZ, EGFP-expressing transplant; 400 islets; n=5; red), and (3) diabetic mice transplanted with PIDO-expressing islets (“Islets^{PIDO+}”; +STZ, PIDO-expressing transplant; 400 islets; n=5; blue). Data are presented as mean±SD. (*P<0.05, **P<0.01, ***P<0.001).

FIG. 5 demonstrates that PIDO-induced immune evasion of engineered islet allografts requires CD4 expression. (A) Schematic of the experiment. PIDO-expressing BALB/c mouse allogeneic islets were transplanted in diabetic CD4-deficient mice. (B) Blood glucose measurements taken before and after STZ treatment and after transplantation of allogeneic islets. Three groups were studied: (1) mice without a transplant (“No Txp”; no STZ, no transplant; black), (2) diabetic mice transplanted with control islets (“Islets^{Ctrl}”; +STZ, EGFP-expressing transplant; red), and (3) diabetic mice transplanted with PIDO-expressing islets (“Islets^{PIDO+}”; +STZ, PIDO-expressing transplant; blue). Data are presented as mean±SEM.

FIG. 6 demonstrates that PIDO-expressing xenogeneic islets survive in immunocompetent murine and canine recipients. (A) Schematic depiction of the experiment. PIDO-expressing porcine islets were transplanted into normoglycemic C57BL/6 mice and dogs. (B) Porcine insulin measurements in normoglycemic immunocompetent C57BL/6 mice after renal subcapsular transplantation with engineered pig islets. Three groups were studied: (1) mice without a transplant (“No Txp”; n=3; black), (2) mice transplanted with control islets (“Islets^{Ctrl}”; EGFP Txp; 400 islets; n=4; red), and (3) mice transplanted with PIDO-expressing islets (“Islets^{PIDO}”; PIDO Txp; 400 islets; n=5; blue). (C) Porcine C-peptide measurements after intravenous glucose tolerance test (GTT) in a normoglycemic beagle dog at 3-, 6-, 10-, 15-, and 20-weeks post-transplantation in epaxial muscle.

FIG. 7 shows a representative western blot comparing IDO expression and abundance in samples from A375 cells that were transduced to express the indicated proteins.

FIG. 8 shows plasmid maps of lentiviral vectors encoding the PIDO fusion protein. (A) Plasmid map of the lentiviral vector comprising an enhanced green fluorescent protein (EGFP) reporter that was used in the Examples. (B) Plasmid map of a lentiviral vector designed for use in a transplant therapy.

### DETAILED DESCRIPTION

A more effective means of inducing immune tolerance would address a critical unmet need to improve the safety of transplantation therapies. To address this unmet need, the inventors created a novel fusion protein, referred to herein as PIDO (PD-L1 and IDO). PIDO comprises peptides derived from two immunoregulatory proteins: programmed death ligand-1 (PD-L1) and indolamine 2,3-dioxygenase (IDO). PD-L1 and IDO are known to induce distinct immune tolerance mechanisms, which are discussed below.

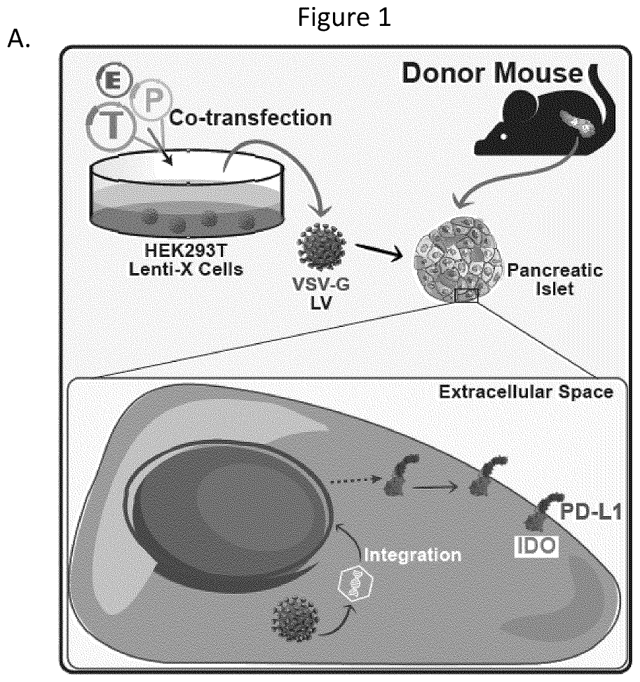

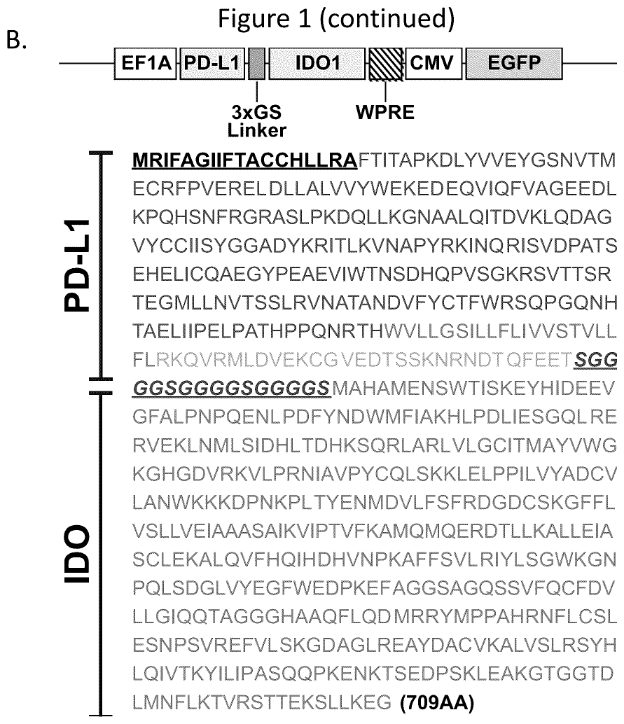

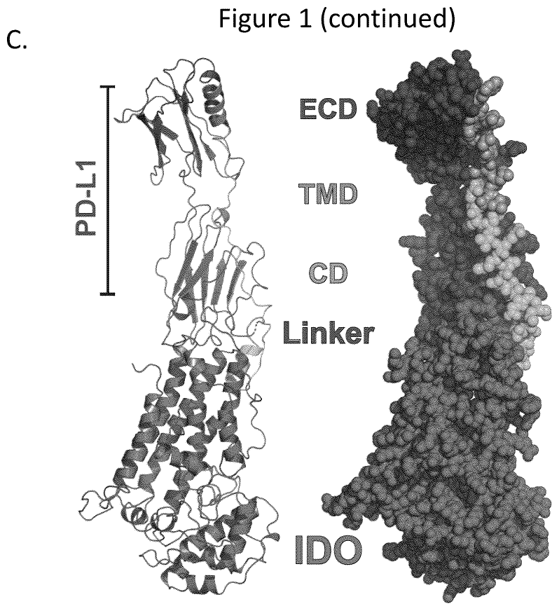

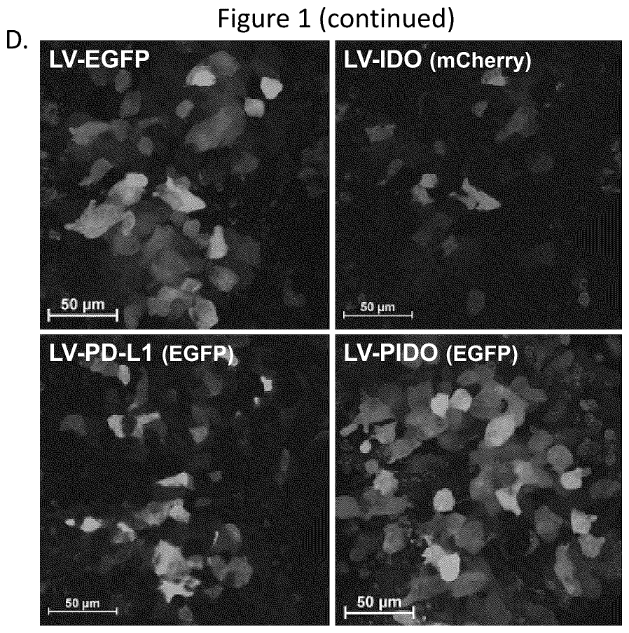

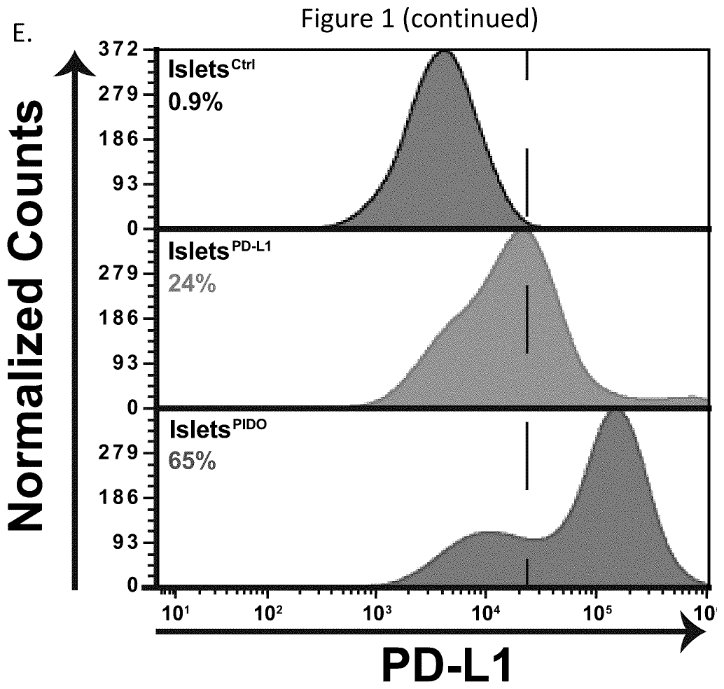

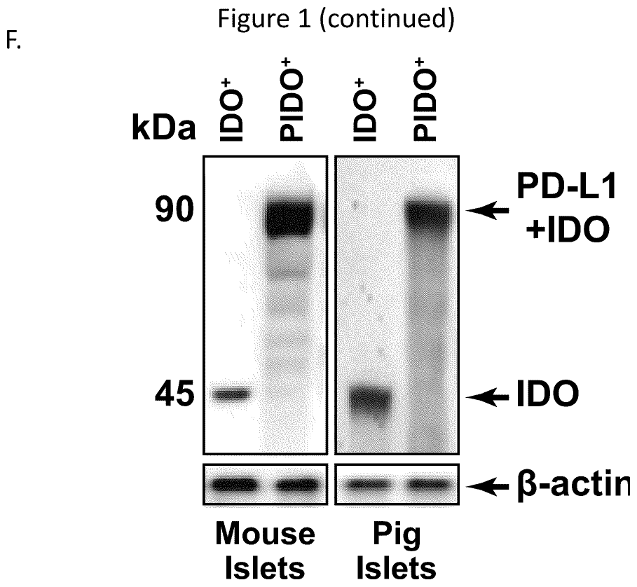

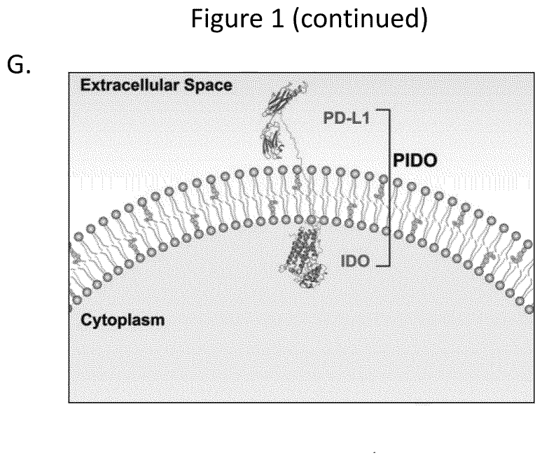

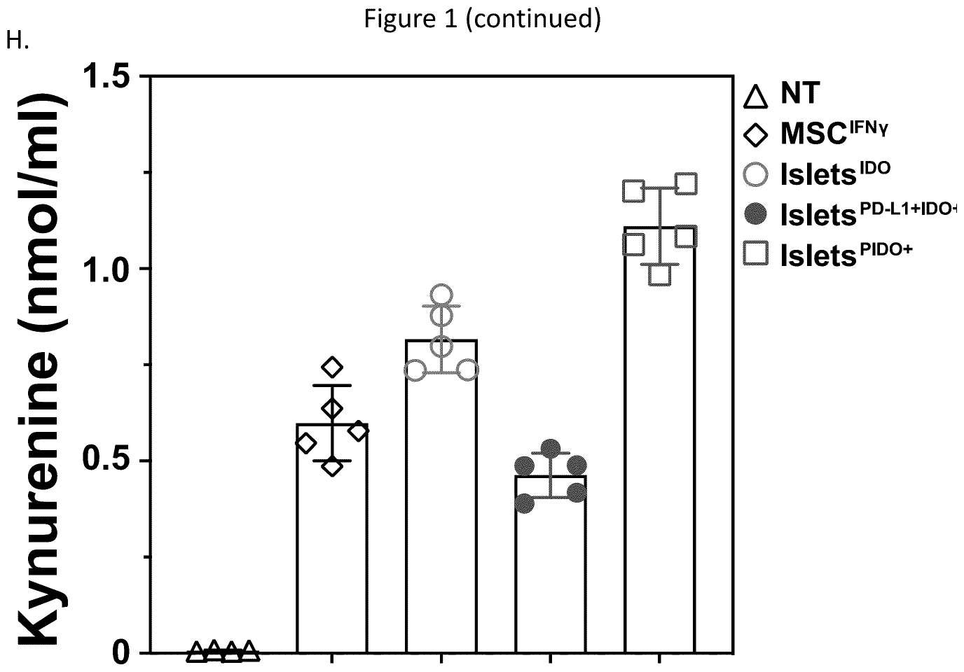

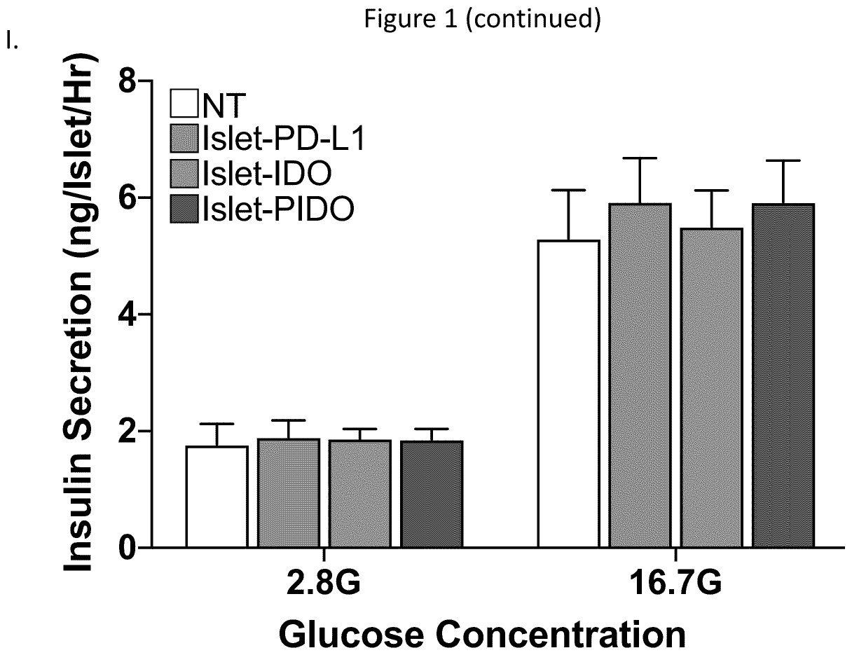

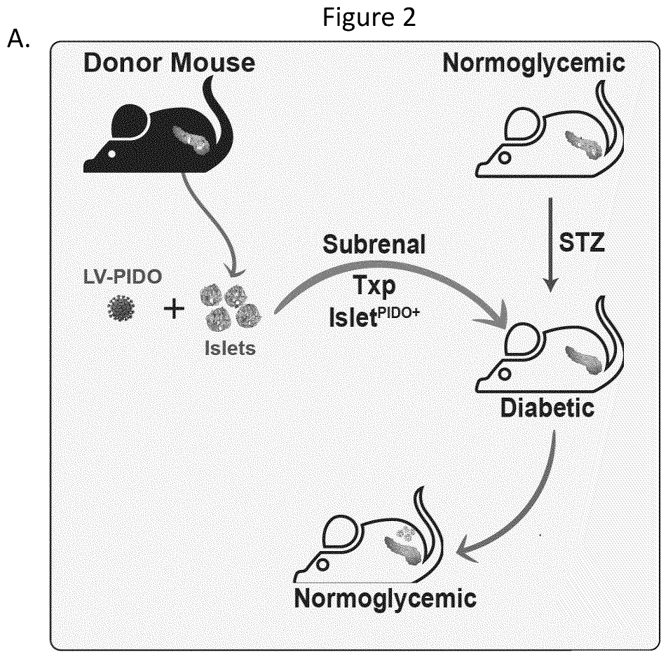

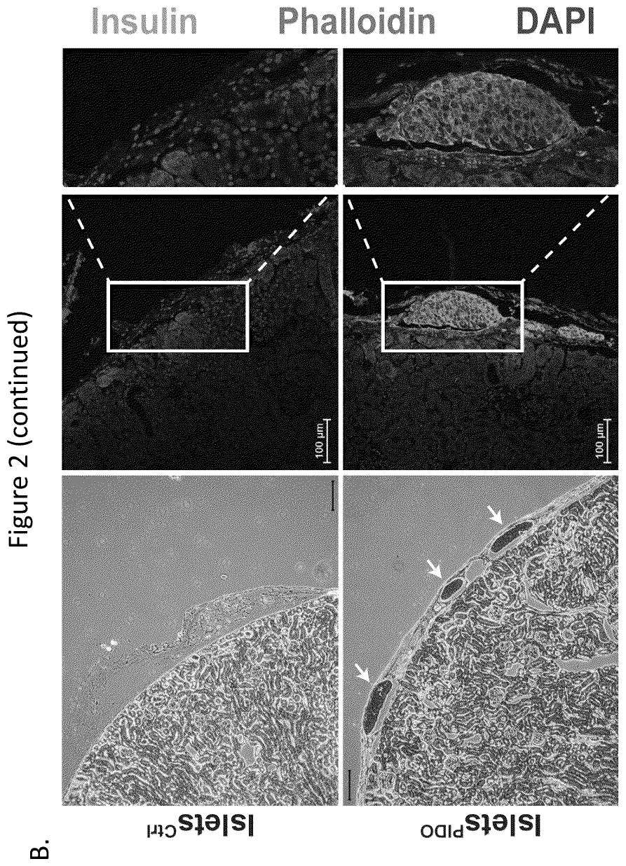

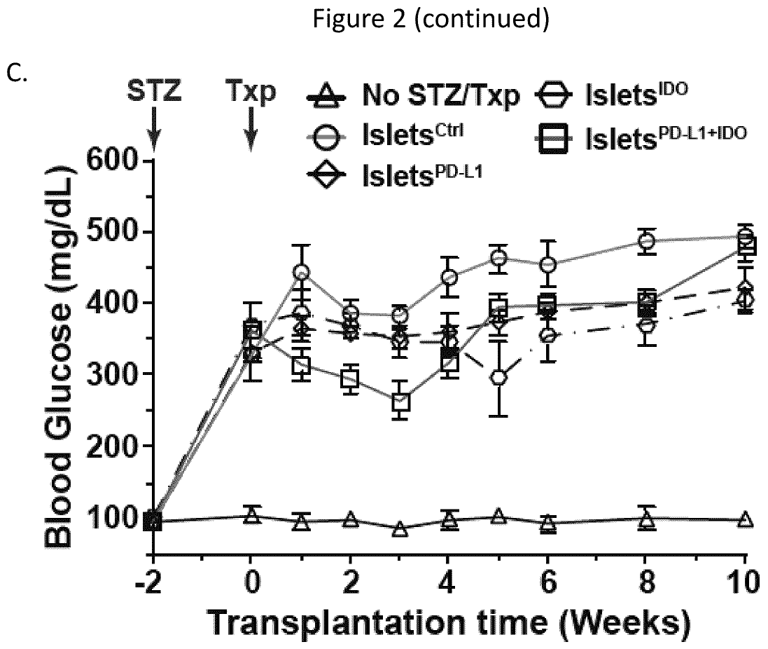

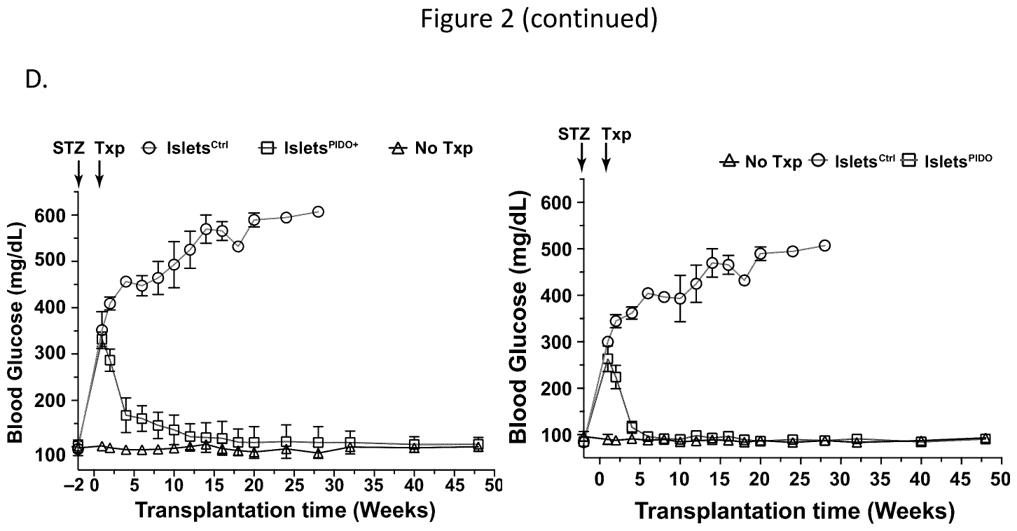

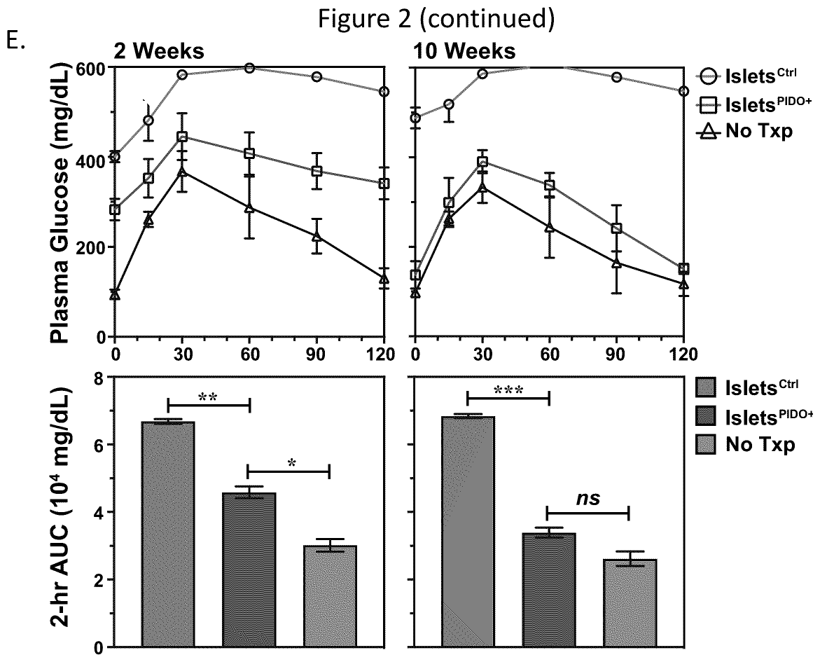

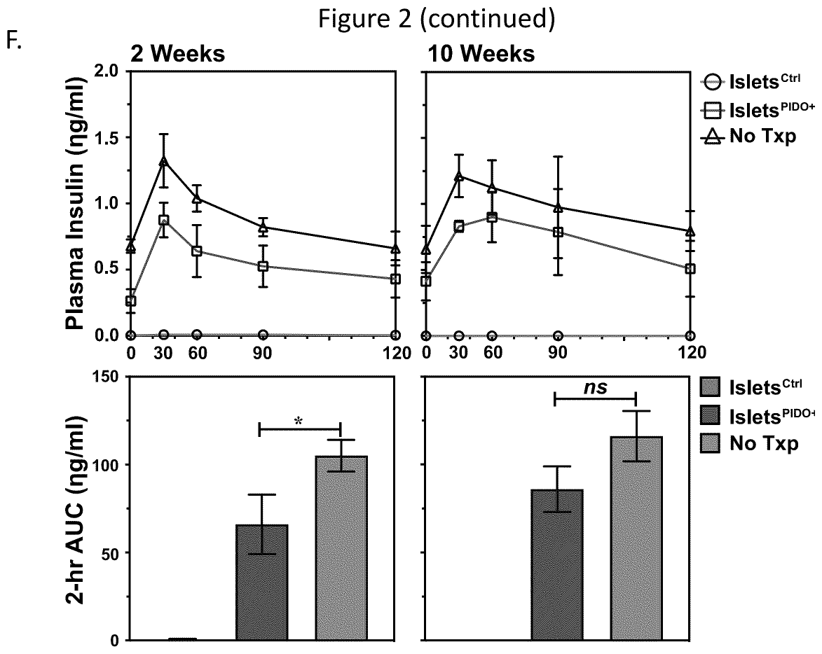

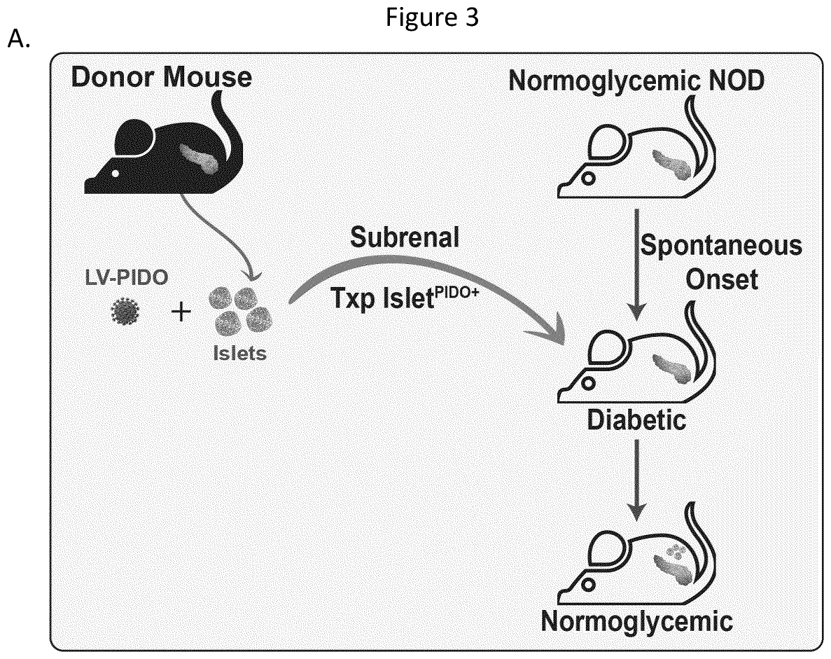

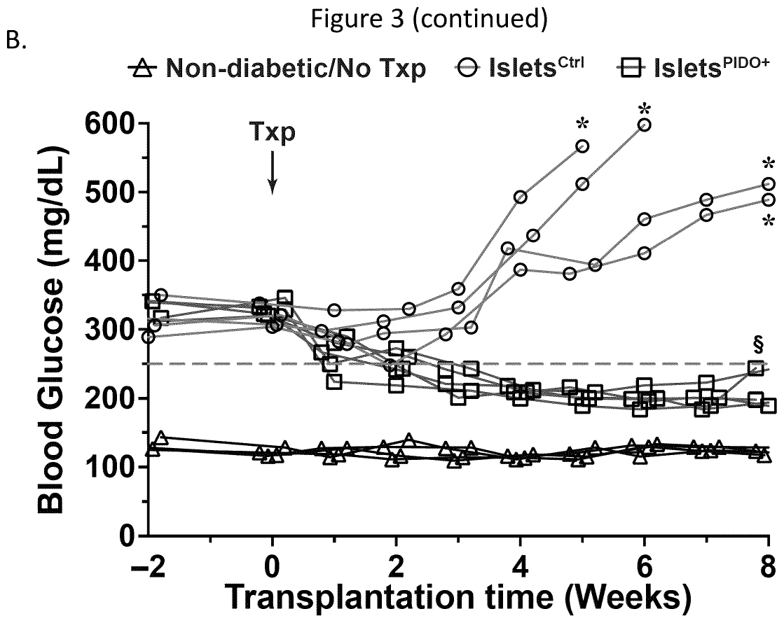

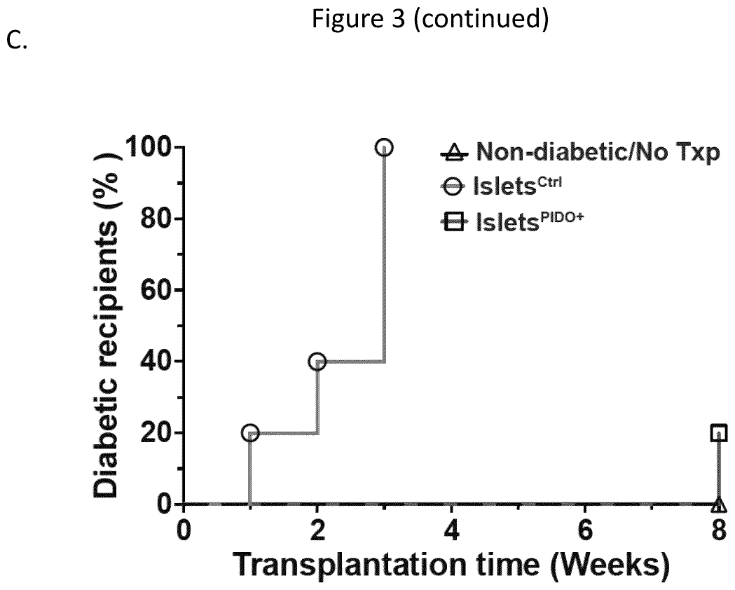

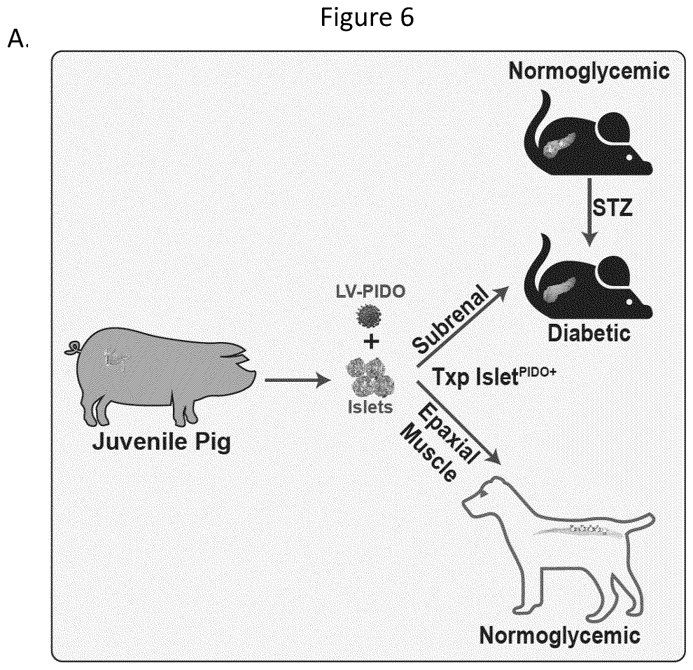

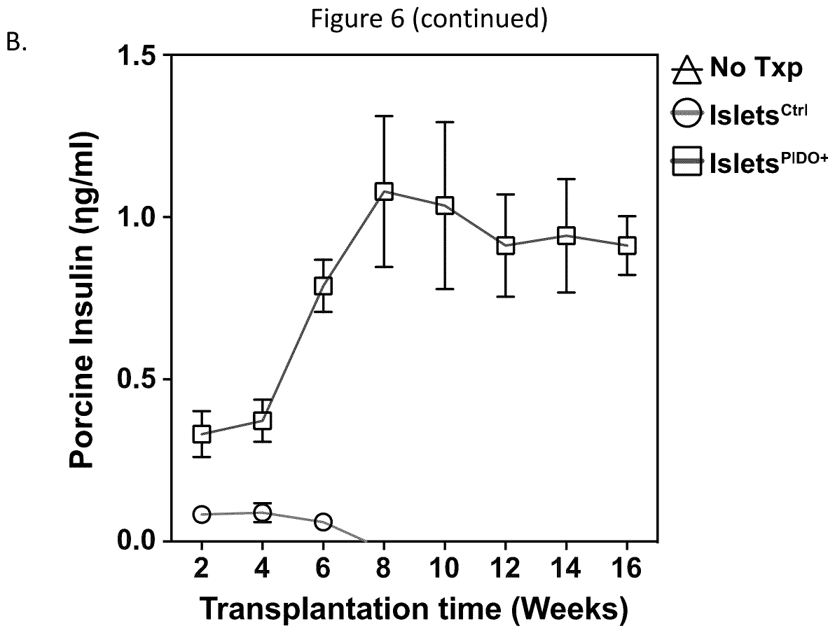

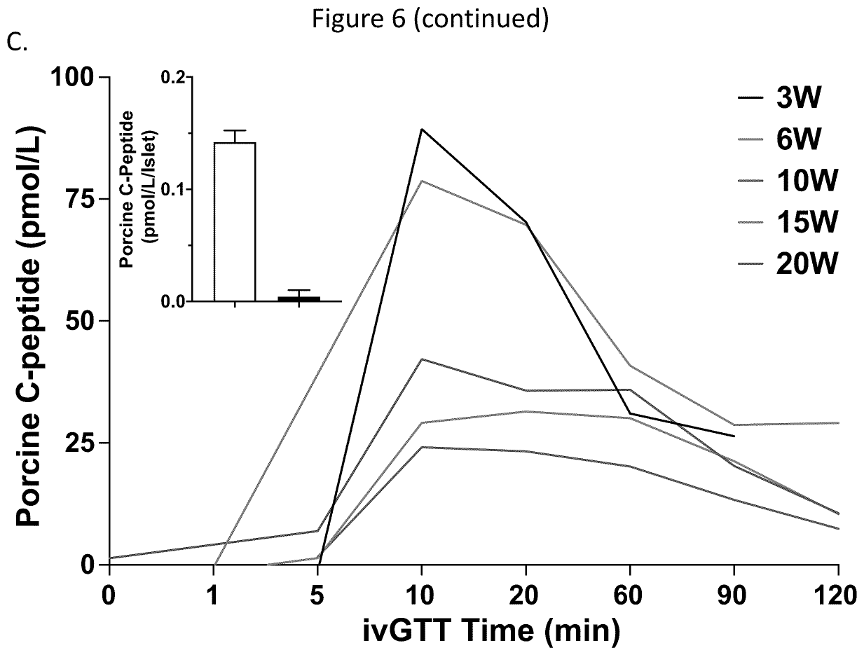

In the Examples, the inventors generate cells that express PIDO and confirm that the components of this fusion protein each localize to the appropriate subcellular compartments (FIG. 1): PD-L1 spans the cell membrane, while IDO is anchored intracellularly via a flexible linker. Further, they confirm that IDO, which usually moves freely throughout the cytoplasm, remains catalytically active when tethered to the membrane as part of this fusion protein (FIG. 1). To test whether the expression of PIDO induces local immune tolerance, the inventors engineered murine pancreatic islets to express this fusion protein and transplanted them into diabetic mice. Following transplantation, the modified islet grafts survived, produced insulin, and reversed the diabetes of these mice (FIG. 2, FIG. 3). Further, the inventors showed that PIDO-expressing porcine islet xenografts remain functional in murine and canine recipients for more than 20 weeks (FIG. 6). Thus, the inventors have demonstrated that expression of the PIDO fusion protein may be used to improve the outcomes of both allogenic and xenogeneic transplant.

The methods of transplanting cells described herein offer multiple advantages over current transplant methods that rely on immune suppression. First, because PIDO remains anchored in the cell membrane, this fusion protein provides immune suppression that is locally restricted. Therefore, the use of PIDO would avoid the undesirable side effects associated with pharmacological immune suppression regimens, which can cause off-target immune suppression and toxicity. Second, the peptide components of PIDO can be matched to the species of the subject for greater compatibility and reduced risk of antigenicity. Third, because nearly all cell types can be modified to express PIDO, this fusion protein can be used with a wide variety of transplantation therapies.

### Fusion Proteins:

In a first aspect, the present invention provides fusion proteins based on the PIDO fusion protein. The fusion proteins comprise, from N-terminus to C-terminus: (a) a PD-L1 peptide comprising at least a portion of the extracellular domain of a PD-L1 protein, (b) a transmembrane domain, and (c) an IDO peptide comprising at least a portion of an IDO protein. Ideally, within the fusion proteins, the PD-L1 peptide is capable of binding to PD-1 and the IDO peptide is catalytically active.

As used herein, the term “fusion protein” refers to a single polypeptide comprising at least two peptide components, e.g., a PD-L1 component and an IDO component. Each peptide component may comprise a synthetic peptide or a naturally occurring peptide. The peptide components may comprise a full-length protein or a fragment thereof, and they may comprise mutations or other modifications relative to the wild-type version of the protein from which they are derived.

Programmed death ligand-1 (PD-L1; also known as cluster of differentiation 274 (CD274)) is a transmembrane protein that plays a major role in suppressing the adaptive immune system. This protein is constitutively expressed by a wide variety of immune cells and can also be expressed by non-immune cells such as pancreatic islets (13, 14). The cognate receptor for this protein, i.e., the programmed cell death-1 (PD-1) receptor, is expressed on the surface of T cells and other immune cells (12). PD-1/PD-L1 binding inhibits effector T cell function and stimulates regulatory T cell function (15, 16). Thus, the PD-1/PD-L1 interaction forms an immune checkpoint that protects normal tissues from inflammation and plays a critical role in the maintenance of immune tolerance.

The PD-L1 peptide used with the present invention must comprise a portion of the extracellular domain of a PD-L1 protein that is capable of binding to PD-1. An “extracellular domain” is a protein domain that localizes to the extracellular space when the protein is expressed by a cell. The amino acid residues within PD-L1 that are necessary for PD-1 binding were recently mapped by Zak et al. (Structure 25(8):1163-1174, 2017), which is incorporated by reference in its entirety. The key residues for PD-1 binding include A121, D122, Y123, K124, and R125 (i.e., the ADYKR sequence). Thus, the PD-L1 peptide used with the present invention should comprise these key amino acid residues. The ability of a PD-L1 peptide to bind to PD-1 may be assessed using a PD1/PD-L1 binding assay or any protein-protein binding assay, including those that utilize surface plasmon resonance, co-immunoprecipitation, or fluorescence resonance energy transfer (FRET). Alternatively, the ability of a PD-L1 peptide to bind to PD-1 may be assessed using in silico modeling.

The PD-L1 peptide may be a portion of a PD-L1 protein from any vertebrate animal. Suitable sources of PD-L1 peptides include, but are not limited to, humans, non-human primates, cows, cats, dogs, pigs, and rodents. In some embodiments, the PD-L1 peptide has at least 95% identity to the extracellular domain of the mouse PD-L1 protein (SEQ ID NO:3; amino acids 19-239 of SEQ ID NO: 2). In other embodiments, the PD-L1 peptide has at least 95% identity to the extracellular domain of the human PD-L1 protein (SEQ ID NO:7).

In some embodiments, the PD-L1 peptide further comprises a PD-L1 signal peptide. The PD-L1 signal peptide is a membrane localization signal that is cleaved off in the mature PD-L1 protein. While the inclusion of a signal peptide is required for proper membrane localization, comparable localization could be achieved by substituting the native PD-L1 signal peptide for the signal peptide of another membrane bound protein or a synthetic signal peptide. In some embodiments, the PD-L1 signal peptide is the signal peptide of the mouse PD-L1 protein (SEQ ID NO:4; amino acids 1-18 of SEQ ID NO: 2). In other embodiments, the PD-L1 signal peptide is the signal peptide of the human PD-L1 protein (SEQ ID NO:8).

A “transmembrane domain” is a protein domain that spans the cell membrane when the protein is expressed by a cell. Transmembrane domains consist predominantly of hydrophobic amino acids. The transmembrane domain of the fusion protein may be any transmembrane domain that does not disrupt the ability of the PD-L1 peptide to bind to PD-1 or the catalytic activity of the IDO protein. In the Examples, the inventors utilized a full-length PD-L1 protein in their PIDO fusion protein, such that both the extracellular domain and the transmembrane domain of the fusion protein were provided by PD-L1. Thus, in some embodiments, the transmembrane domain comprises at least a portion of the transmembrane domain of a PD-L1 protein. In some embodiments, the transmembrane domain has at least 95% identity to the transmembrane domain of the mouse PD-L1 protein (SEQ ID NO:5). In other embodiments, the transmembrane domain has at least 95% identity to the transmembrane domain of the human PD-L1 protein (SEQ ID NO:9).

Indolamine 2,3-dioxygenase (IDO) is an intracellular, heme-containing enzyme that catalyzes the oxidation of tryptophan. This enzyme performs the initial, rate-limiting step necessary to degrade tryptophan via the kynurenine pathway. Tryptophan degradation and the products of this process (i.e., kynurenine derivatives and 02 free radicals) suppress innate and adaptive immunity by several mechanisms, including apoptosis, inhibition of activated T cells, and activation of resting regulatory T cells (19). IDO can be expressed in a variety of human tissues when its expression is induced by inflammatory cytokines, and it is known to be expressed in chronic inflammatory conditions such as cancers, infections, autoimmune and allergic diseases, and transplant rejection (20). Further, recent reports suggest that subsets of human myeloid dendritic cells and cancer cells constitutively express IDO to suppress allogeneic T-cell immune responses (21, 22).

The IDO peptide used with the present invention must comprise a catalytically active portion of an IDO protein, i.e., a portion that can catalyze 1-tryptophan oxidation. Sugimoto, et al. (Proc Natl Acad Sci USA (2006), 103(8): 2611-2616) have determined that amino acid residues F226, F227, and R231 of IDO are essential for its catalytic activity. Thus, the IDO peptide used with the present invention should comprise these key residues. The catalytic activity of the IDO peptide may be assessed by measuring conversion of tryptophan to kynurenine, for example, by kynurenine ELISA.

The IDO peptide may be a portion of an IDO protein from any vertebrate animal. Suitable animals include, but are not limited to, humans, non-human primates, cows, cats, dogs, pigs, and rodents. In some embodiments, the IDO peptide has at least 95% identity to the full-length human IDO protein (SEQ ID NO:10).

In some embodiments, the transmembrane domain is linked to the IDO peptide by a linker peptide. As used herein, the term “linker peptide” refers to a peptide that connects two peptide components within a fusion protein. The linker may be flexible such that it has no fixed structure in solution and the adjacent peptide components are free to move relative to one another. The flexible linker comprises 1 or more amino acid residues, preferably 1, 2, 3, 4, 5, 6, 7, 8, 9, 10, 11, 12, 13, 14, 15, 16, 17, 18, 19, or 20 or more residues. The linker may be an existing sequence provided by a protein included in the fusion protein or it may be provided by insertion of one or more amino acid residues between the peptide components of the fusion protein. The linker may comprise any amino acid sequence that does not substantially hinder the function of the peptide components (i.e., PD-L1's ability to bind PD-1 and IDO's catalytic activity). Preferred amino acid residues for flexible linker sequences include glycine, alanine, serine, threonine, lysine, arginine, glutamine, and glutamic acid, but are not limited thereto. In some embodiments, the linker peptide is a glycine-serine linker (i.e., a linker consisting of serine and glycine). In specific embodiments, the glycine-serine linker is a 3×GGGS linker (SEQ ID NO:11).

In some embodiments, the fusion protein comprises the mouse PIDO fusion protein described in the Examples (SEQ ID NO:1; encoded by SEQ ID NO:12), which comprises the full-length mouse PD-L1 protein (SEQ ID NO:2) linked to the full-length human IDO protein (SEQ ID NO:10) via a 3×GGGS linker (SEQ ID NO:11). In other embodiments, the fusion protein comprises the human PIDO fusion protein (SEQ ID NO:14; encoded by SEQ ID NO:15), which comprises the full-length human PD-L1 protein (SEQ ID NO:6) linked to the full-length human IDO protein (SEQ ID NO:10) via a 3×GGGS linker (SEQ ID NO: 11). In other embodiments, the fusion protein comprises the canine PIDO fusion protein (SEQ ID NO:17; encoded by SEQ ID NO:18), which comprises the full-length canine PD-L1 protein (SEQ ID NO:23) linked to the full-length human IDO protein (SEQ ID NO:10) via a 3×GGGS linker (SEQ ID NO:11). In other embodiments, the fusion protein comprises the feline PIDO fusion protein (SEQ ID NO:20; encoded by SEQ ID NO:21), which comprises the full-length feline PD-L1 protein (SEQ ID NO:24) linked to the full-length feline IDO protein (SEQ ID NO:10) via a 3×GGGS linker (SEQ ID NO:11).

### Nucleic Acid Constructs:

The present invention provides nucleic acid constructs comprising a polynucleotide encoding the fusion proteins described herein operably linked to a promoter.

The terms “polynucleotide,” “oligonucleotide,” and “nucleic acid” are used interchangeably to refer a polymer of DNA or RNA. A polynucleotide may be single-stranded or double-stranded and may represent the sense or the antisense strand. A polynucleotide may be synthesized or obtained from a natural source. A polynucleotide may contain natural, non-natural, or altered nucleotides, as well as natural, non-natural, or altered internucleotide linkages. The term polynucleotide encompasses constructs, plasmids, vectors, and the like.

As used herein, the term “construct” or “nucleic acid construct” refers a to recombinant polynucleotide, i.e., a polynucleotide that was formed by combining at least two polynucleotide components from different sources, natural or synthetic. For example, a construct may comprise the coding region of one gene operably linked to a promoter that is (1) associated with another gene found within the same genome, (2) from the genome of a different species, or (3) is synthetic. Constructs can be generated using conventional recombinant DNA methods.

In some embodiments, the nucleic acid construct is a viral vector. As used herein, a “viral vector” is a recombinant viral nucleic acid that has been engineered to express a heterologous polypeptide (e.g., the fusions proteins of the present invention). Viral vectors include cis-acting elements that drive the expression of the encoded heterologous polypeptide. Suitable viral vectors are known in the art and include, but are not limited to, adenovirus vectors; adeno-associated virus vectors, pox virus vectors (e.g., fowlpox virus vectors), alpha virus vectors, baculoviral vectors, herpes virus vectors, retrovirus vectors (e.g., lentivirus vectors), Modified Vaccinia virus Ankara vectors, Ross River virus vectors, Sindbis virus vectors, Semliki Forest virus vectors, and Venezuelan Equine Encephalitis virus vectors. In a preferred embodiment, the viral vector is a lentiviral vector.

As used herein, the term “promoter” refers to a DNA sequence that regulates the expression of a gene. Typically, a promoter is a regulatory region that is capable of binding RNA polymerase and initiating transcription of a downstream (3′ direction) sequence. However, a promoter may be located at the 5′ or 3′ end, within a coding region, or within an intron of a gene that it regulates. Promoters may be derived in their entirety from a native gene, may be composed of elements derived from multiple regulatory sequences found in nature, or may comprise synthetic DNA. A promoter is “operably linked” to a polynucleotide if the promoter is connected to the polynucleotide such that it can affect transcription of the polynucleotide. It is understood by those skilled in the art that different promoters may direct the expression of a gene in different tissues or cell types, at different stages of development, or in response to different environmental conditions. Suitable promoters for use with the present invention include, but are not limited to, constitutive, inducible, temporally regulated, developmentally regulated, chemically regulated, tissue-preferred, and tissue-specific promoters. In some embodiments, the promoter is an elongation factor 1α short (EFS) promoter or a hybrid CMV enhancer/chicken β-actin (CBA) promoter. The EF-1α promoter is known to be one of the strongest promoters for driving expression in various mammalian cell lines. The CBA promoter is commonly used for gene transfer because it provides robust, long-term expression in all cell types. Those of skill in the art will understand how to select an appropriate promoter to drive expression of the fusion proteins disclosed herein for a particular application.

In some embodiments, the nucleic acid construct is SEQ ID NO:13, i.e., a lentiviral vector encoding the PIDO fusion protein comprising mouse PD-L1 (SEQ ID NO:1). In some embodiments, the nucleic acid construct is SEQ ID NO:16, i.e., a lentiviral vector encoding the PIDO fusion protein comprising human PD-L1 (SEQ ID NO:14). In some embodiments, the nucleic acid construct is SEQ ID NO:19, i.e., a lentiviral vector encoding the PIDO fusion protein comprising canine PD-L1 (SEQ ID NO:17). In some embodiments, the nucleic acid construct is SEQ ID NO:22, i.e., a lentiviral vector encoding the PIDO fusion protein comprising feline PD-L1 (SEQ ID NO:20).

### Cells:

The present invention provides cells comprising the nucleic acid construct described herein. Under suitable conditions, the cells express the fusion proteins described herein.

A “cell” is the basic unit from which all living things are composed. Every cell consists of cytoplasm (i.e., gelatinous liquid that fills the inside of the cell) enclosed within a membrane. The space outside of the cell membrane is referred to as the “extracellular space”.

Any cell type may be used with the present invention. In some embodiments, the cell is useful for transplantation. For example, in some embodiments, the cell is an induced pluripotent stem cell, embryonic stem cell, retinal pigment epithelial cell, dopaminergic neuron, stromal cell, or cardiomyocyte. In certain embodiments, the cell is a hematopoietic stem cell or mesenchymal stem cell. In the Examples, the inventors generated islets that express the PIDO fusion protein. Thus, in preferred embodiments, the cells are islets i.e., pancreatic cells that produces hormones (e.g., insulin and glucagon) that are secreted into the bloodstream.

In some embodiments, the nucleic acid construct is a viral vector, and the nucleic acid construct is introduced to the cell by viral infection. In other embodiments, the nucleic acid construct is introduced to the cell using plasmid DNA, transposons, CRISPR-based gene editing, or chromosome transfer.

The inventors designed the PIDO fusion protein such that (1) the PD-L1 extracellular domain would localize to the extracellular space where it can interact with PD-1 receptors on the surface of activated T cells, and (2) the IDO protein would localize to the cytoplasm where it can function in the kynurenine pathway. Thus, in some embodiments, at least a portion of the fusion protein is expressed on the surface of the cell. In preferred embodiments, the PD-L1 peptide is localized in the extracellular space and the IDO peptide is localized in the cytoplasm of the cell.

Any method of protein detection may be used to test whether a cell expresses a fusion protein disclosed herein. Suitable methods for detecting proteins include, without limitation, enzyme-linked immunoassay (ELISA), dot blotting, western blotting, flow cytometry, mass spectrometry, and chromatographic methods. In the Examples, PD-L1 was detected at the cell surface via flow cytometry using an anti-CD274 antibody, whereas IDO was detected intracellularly via western blot (FIG. 1). Thus, in certain embodiments, the fusion protein is detected using flow cytometry or western blot.

### Methods:

The present invention provides methods of transplanting a cell described herein into a subject. As used herein, the term “transplanting” refers to a procedure in which cells from a donor are placed in the body of a recipient. The transplant may be allogeneic, i.e., from a different individual of the same species, or xenogeneic, i.e., from an individual of a different species. The methods may involve any transplant techniques known in the art. The transplanted cells may be individual cells. Alternatively, the transplanted cells may be part of an organ, tissue, organoid, or cellular aggregate. Importantly, these methods will allow treatments that rely upon cells that are in limited supply (e.g., islets from human cadavers) to be replaced with treatments that utilize cells from a renewable source (e.g., embryonic stem cells).

The transplanted cells may be from any suitable donor. Suitable donor animals include, but are not limited to, humans, non-human primates, cows, cats, dogs, pigs, and rodents. The donor cells may be from an allogenic or xenogeneic source. For example, for a human recipient, the donor cells may from another human (i.e., an allogenic source) or a pig (i.e., a xenogeneic source). Suitable xenogeneic sources for transplant into humans include mammalian sources such as pigs, sheep, cows, horses, and non-human primates. Because humans are known to respond to pig insulin, pigs are a promising source of pancreatic islets for transplantation into type I diabetics. Thus, in some embodiments, the transplanted cells are from a pig.

The “subject” (i.e., recipient) may be any animal that could reasonably receive transplant cells from the donor. Suitable subjects include, but are not limited to, humans, non-human primates, cows, cats, dogs, pigs, and rodents. In some embodiments, the subject is a human. In some embodiments, the subject is in need of a functional cell or tissue. For example, in some embodiments, the subject has diabetes and is in need of functional islets.

Advantageously, the fusion protein, particularly the extracellular PD-L1 peptide portion, is matched to the species of the subject for greater compatibility and reduced risk of antigenicity. However, those of skill in the art will understand that matching the species is less critical for proteins that are highly conserved (e.g., IDO) as compared to those that are less conserved (e.g., PD-L1).

In the absence of immunosuppression, allogenic and xenogeneic transplants are destroyed by the recipient's immune system, which attacks the transplants as a foreign substance. However, in the Examples, the inventors demonstrate that expression of the PIDO fusion protein by transplanted cells locally suppresses the immune system. Specifically, they demonstrate that PIDO-expressing murine islets transplanted into mice (i.e., an allogenic graft; see FIG. 2) and PIDO-expressing porcine islets transplanted into mice and dogs (i.e., a xenogeneic graft; see FIG. 6) survive and are functional in the recipient animal. Thus, in some embodiments, the transplanted cell is tolerated by the immune system in the absence of immunosuppression. A transplanted cell is “tolerated” when the immune system of the recipient is unresponsive or minimally responsive to it. Immune tolerance can be assessed by monitoring the survival or function of the transplanted cells. For example, the inventors showed that the transplanted PIDO-expressing porcine islets survived longer than naïve porcine islets (i.e., islets that were not engineered to express PIDO) and remained functional (i.e., produced insulin) in recipients for more than 20 weeks. Thus, in some embodiments, the transplanted cells may exhibit prolonged survival relative to a transplanted control cell lacking the nucleic acid construct encoding the fusion protein. Alternatively, immune tolerance may be inferred by a lack of immune rejection (i.e., by quantifying the number of reactive immune cells that co-localize with PIDO-expressing grafts) or by the presence of regulatory T cells, which mediate immune tolerance.

As used herein, the term “immunosuppression” refers to the partial or complete suppression of the immune response of a subject. Immunosuppression may be deliberately induced in a subject using drugs to help transplanted donor cells survive. Examples of immunosuppressive drugs that are used to reduce the risk of transplant rejection include, without limitation, tacrolimus, cyclosporine, mycophenolate mofetil, azathioprine, everolimus, sirolimus, and glucocorticoids (steroids).

The cells that are transplanted in the methods of the present invention may be of any cell type that is amenable to ex vivo transplantation. In some embodiments, the transplanted cell performs its native function (e.g., an islet produces insulin).

In the Examples, the inventors engineered allogeneic islets to express the PIDO fusion protein and transplanted them into immune competent diabetic mice. Thus, in some embodiments, the subject is diabetic, and the cell is an islet. Diabetes mellitus, commonly known as diabetes, is a group of metabolic disorders that is characterized by a high blood sugar level (hyperglycemia) over a prolonged period. There are three main types of diabetes: type 1 diabetes, type 2 diabetes, and gestational diabetes. Type 1 diabetes results from the failure of the pancreas to produce enough insulin due to the destruction of insulin-producing pancreatic beta cells by a beta cell-specific autoimmune process. Type 2 diabetes is caused by insulin resistance, a condition in which cells fail to respond to insulin properly. Type 2 diabetes primarily occurs as a result of obesity and lack of exercise. Gestational diabetes occurs when pregnant women without a previous history of diabetes develop high blood sugar levels.

Ideally, the diabetic subject treated by the present methods will produce insulin post-transplantation with PIDO-expressing islets. Insulin secretion can be measured, for example, using the glucose-stimulated insulin secretion (GSIS) test. In the GSIS test, blood is sampled at specific time points to measure plasma insulin levels in the basal (fasted) state and after induction of hyperglycemia via administration of a glucose bolus. Alternatively, insulin secretion can be measured indirectly via detection of C-peptide, a protein that is produced and secreted along with insulin. C-peptide tests are commonly used by doctors to diagnose type I diabetes.

Additionally, diabetic subjects treated by the present methods may demonstrate improved glucose tolerance post-transplantation as compared to pre-transplantation. Glucose tolerance can be measured using any glucose tolerance test known in the art. Alternatively, glycosylated hemoglobin (HbAlc) may be measured as an indicator of long-term glycemic control.

In some embodiments, the subject becomes normoglycemic post-transplantation. As used herein the term “normoglycemic” refers to the presence a normal concentration of glucose in the blood. The concentration of glucose in the blood can be measured using any blood glucose test. A blood glucose level of less than 140 mg/dL is considered normal in humans, whereas, in mice, a blood glucose level of less than 100 mg/dL is considered normal. However, fed mice with less than 200 mg/dL blood glucose are also considered non-diabetic or normoglycemic. In some embodiments, the subject remains normoglycemic for at least 50 weeks post-transplantation.

In other embodiments, the cells used in the methods of the present invention are derived from stem cells. Suitable stem cells for use with the present invention include, without limitation, embryonic stem cells (ESC), induced pluripotent stem cells (iPSC), hematopoietic stem cells (HSC), and mesenchymal stem cells (MSC). In certain embodiments, the cell is the differentiated progeny of a hematopoietic stem cell, which give rise to myeloid, lymphoid, and monocytic cell types. The stem cells may be transplanted into the animal in an undifferentiated state or may be differentiated in vitro prior to transplantation. Stem cells may be obtained from established stem cell lines or may be obtained directly from primary tissue.

The inventors also envision that the fusion proteins of the present invention could be used to generate genetically modified transplant donor animals. For example, pigs could be genetically engineered to express the PIDO fusion protein throughout their bodies, such that they produce whole organs and tissues that could be used as xenogeneic transplants for humans. Suitable organs for transplantation include, without limitation, kidney, heart, liver, lungs, pancreas, intestine, thymus, and uterus. Suitable tissues for transplantation include, for example, bones, tendons, corneae, skin, heart valves, nerves, and veins.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

Percent identity (% sequence identity or % identity). Refers to the percentage of residue matches between at least two amino acid sequences aligned using a standardized algorithm. Methods of amino acid sequence alignment are well known in the art. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases. Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 10, at least 15, at least 20, or more contiguous residues. Such lengths are exemplary only, and it is understood that any fragment length supported by the sequences shown herein, in the tables, figures or Sequence Listing, may be used to describe a length over which percentage identity may be measured.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### Examples

Allogeneic islet transplantation is a promising experimental therapy for poorly controlled diabetes but is limited by the adverse effects of chronic immunosuppression. Induction of immune tolerance against allogeneic antigens is necessary to prevent allograft rejection and to obviate the need for immunosuppressant drugs. However, the need for an effective means to induce immune tolerance remains unmet.

In the following example, the inventors describe a novel fusion protein that was created by combining two biochemically distinct proteins: programmed death ligand-1 (PD-L1) and indoleamine 2,3-dioxygenase (IDO). PD-L1 is a transmembrane protein that is known to play a major role in suppressing the adaptive immune system. IDO is an intracellular, monomeric, heme-containing enzyme that regulates the breakdown of tryptophan in the kynurenine pathway. IDO affects immune tolerance by regulating the function of natural killers (NK), T cells, T regulatory cells (Tregs) and myeloid-derived suppressor cells (MDSC) via tryptophan depletion. Thus, the inventors' fusion portion, which is referred to herein as PIDO (PD-L1+IDO), offers two distinct tolerogenic mechanisms for the prevention of transplant rejection.

The inventors have demonstrated that PIDO is robustly expressed in and displayed on the surface of mammalian cells, including mouse and pig islets. When allogeneic PIDO-expressing islets are transplanted into hyperglycemic mice, the islet grafts survive and reverse both streptozotocin-induced and autoimmune diabetes for more than 50 weeks and 10 weeks, respectively. Further, PIDO-expressing porcine islet xenografts exhibit glucose-responsive insulin secretion for up to 30 weeks in euglycemic dogs. The survival of these PIDO-expressing allografts and xenografts suggests that this fusion protein may be a means to achieve local immunomodulation and allow for improved transplant outcomes in the absence of chronic immunosuppression.

### Materials and Methods:

Study design. The objective of this study was to generate allogenic PIDO-expressing islets and transplant them into mice with preexisting diabetes to test the ability of the PIDO fusion protein to induce immune tolerance to the allogeneic islets. We used lentiviral delivery to genetically engineer islets derived from allogeneic or xenogeneic donors. We transplanted PIDO-expressing islets and naïve islets (i.e., islets that were not engineered to express PIDO) into 15 and 9 streptozotocin (STZ)-treated diabetic mice, respectively. STZ-treated diabetic and nondiabetic mice without transplants served as transplantation controls. Mouse groups were assigned randomly, and the study was not blinded. Transplanted mice were monitored through blood glucose measurements and blood plasma collection and were then euthanized for ex vivo analysis. Nephrectomy surgery was performed on PIDO* islet transplanted mice to confirm that the transplanted islets were the source of the glucose tolerance and nondiabetic blood glucose concentrations observed in these mice. Data collection was stopped at predetermined, arbitrary times. Mice that did not develop diabetes post-STZ administration and mice that died pre- or peri-transplant surgery were excluded from the study.

Enzymatic activity of IDO1. Kynurenine levels were analyzed in conditioned media collected from mesenchymal stromal cells (positive control) or islets via enzyme-linked immunosorbent assay (ELISA) using the Kynurenine ELISA kit (#F56401, LSBio, USA) 48 hours after the cells were transduced with a PIDO-encoding lentiviral vector.

Glucose-stimulated insulin secretion (GSIS). To assess static GSIS, approximately 50 size-matched islets were transduced with lentiviral vectors encoding either PIDO or EGFP (control) in 48-well plates. Islets were washed with KRB buffer and were then pre-incubated in glucose-free KRB buffer for 30 minutes. Static insulin secretion was measured by incubating islets in media with basal (2.8 mM or 2.8 G) or stimulatory (16.7 mM or 16.7 G) glucose for 2 hours each. The supernatant was collected for use in an insulin assay. To perform intracellular insulin detection, the islets were harvested, rinsed with PBS, resuspended in 300 μL acid ethanol, and homogenized by ultrasonic disruption of the cell membrane. Insulin was measured using a mouse insulin ELISA kit (#10-1247-01, Mercodia, Uppsala, Sweden) according to the manufacturer's protocol.

Immunocytochemical staining and imaging. Intact mouse islets were transduced with lentivirus vectors that delivered various transgenes (EGFP, PD-L1:EGFP, IDO:mCherry, and PIDO:EGFP) and were stained with nuclear counterstain Hoechst 33342 (Cat #H1399, ThermoFisher, USA). Formalin-fixed paraffin embedded kidney sections from recipient mice were either stained with hematoxylin-eosin (H&E) for visualization of islet microscopic anatomy or with anti-insulin antibody (1:1000; Immunostar, USA) and actin (Acti-Stain 555 Phalloidin, Cat #PHDH1) for detection of transplanted, insulin-positive islets by immunofluorescence (IF) microscopy and imaging. Nuclei were counterstained with ProLong™ Diamond Antifade Mountant (#P36970, ThermoFisher, USA). H&E images were acquired using a Zeiss AX10 inverted microscope equipped with a Zeiss Axiocam 305 color camera. IF images were acquired using a laser-scanning microscope (A1R; Nikon, USA).

Islet cell flow cytometry. Islets expressing the PIDO fusion protein, PD-L1 only, or EGFP (control) were washed in 2 mmol/l EDTA/PBS, incubated for 5 minutes at ambient temperature in Ca²⁺-free PBS supplemented with 0.025% trypsin, and dissociated into a single-cell suspension by gentle pipetting. Dissociated islets were stained with viability dye (Ghost Dye Red 780, Cat #13-0865, Tonbo Biosciences, USA) for 30 minutes, and were then stained for CD274 (PD-L1) to detect PD-L1 expression on the cell membrane. PD-L1 and EGFP stained cells were used to set the gates. All samples were FSC-H and SSC-H gated and then FSC-A/FSC-H gated to select single cells. Live cells were gated based on Ghost Red 780. Flow cytometry plots for PD-L1 expression are shown as histograms.

Islet isolation and culture. Juvenile porcine islets were isolated from the pancreata of 8- to 15-day-old, pre-weaned Yorkshire piglets and were cultured as described previously (52). Mouse islets were isolated from male 12- to 16-week-old C57BL/6J mice (Jackson Laboratory, USA) as described previously (53). Islets were cultured (37° C., 5% CO₂) in RPMI-1640 medium (Corning, USA) with 10% FBS (Gibco, USA) and 1% antibiotic-antimycotic (ThermoFisher, #15240096) for the indicated duration or overnight before they were co-cultured with pluripotent stem cells (PSCs) in a 1:1 mix of complete RPMI and DMEM F-12 (RD^{mix}) media.

Lentiviral transduction of mouse and pig pancreatic islets. After islet viability was assessed using dithiazine, islets were cultured in RPMI medium overnight. The next day, islets were partially disrupted by mild enzymatic dissociation. Briefly, islets were incubated for two minutes in pre-warmed Accutase (2.5 ul/islet, StemCell technologies) and washed with Ca/Mg-free HBSS. Purified viruses were added to the islets in an ultra-low attachment plate or dish (Costar, Corning) and were incubated with viral supernatant for 6 hours or overnight. For the default transduction condition, a vesicular stomatitis virus glycoprotein (VSV-G)-pseudotyped cytomegalovirus-green fluorescent protein (CMV-GFP) vector was used at a multiplicity of infection (MOI) of 10, and transduction was performed in serum-free medium supplemented with 0.1% bovine albumin, 1× Insulin-Transferrin-Selenium (ITS) (Sigma Aldrich), and 8 ug/ml polybrene. The transduction volume was kept uniform throughout all experiments. The volume of the growth area of the well/dish was 135.5 μl/cm² and a minimum of 50% of the transduction volume consisted of fresh medium. Islets were cultured in RPMI medium supplemented with 10% FBS for 48 hours, and the transduction efficiency was evaluated prior to transplant.

Mouse transplants. Mice were randomly designated for the STZ treatment and transplantation groups. The number of mice per group (i.e., 9 and 15) was selected to allow for statistical significance. Surgical procedures and follow-up studies were performed by unblinded individuals. Male ˜8-week-old BALB/c, C57BL6/j, and CD4^{-/-} (B6.129S2-Cd4^{tm1Mak}/J, Strain #002663) mice were purchased from the Jackson Laboratory and were rendered diabetic via injection of STZ (45 mg/kg; R&D systems) for 5 days. Diabetes was confirmed after 7 days. Spontaneously diabetic female NOD mice (˜12-16 weeks old) with blood glucose levels higher than 350 mg/dl were transplanted with islets harvested from euglycemic 8-week-old C57BL/6J donor mice. Anaesthetized mice were transplanted with ˜400 handpicked, mixed size islets (PIDO-expressing or control transduced), or saline under the kidney capsule. Animals were monitored for up to 50 weeks. Blood glucose was measured with a Contour Blood Glucose Monitoring System (Bayer). Glucose tolerance and in vivo GSIS assays were performed by fasting mice for 4 hours and then injecting them with glucose (2 g/kg). Serum hormones were quantified using ELISA kits for insulin (mouse #10-1247-01, porcine #10-1200-01) and porcine C-peptide (#10-1256-01) following the manufacturer's instructions (Mercodia, Uppsala, Sweden). Twenty weeks after transplantation, transplant recipient mice were rechallenged, either by a second STZ injection or by live nephrectomy, which was performed on five anaesthetized mice from each group.

Dog transplants. An intact male beagle (10 kg) was used in this study. The dog was sedated and anesthetized using approved agents. Anesthesia was maintained by inhalation of isoflurane (0.75-1.75%) in oxygen. Carprofen (4.4 mg/kg; Rimadyl®, Zoetis, Parsippany, NJ) was given subcutaneously at the time of anesthesia and on the day after implantation of cells to provide analgesia. The skin overlying the epaxial musculature of the back was prepared for aseptic surgery removing the hair and scrubbing with chlorhexidine from the thirteenth rib to the cranial limit of the ileal crest. A small (5 mm) stab incision was made in the skin 2 cm caudal to the thirteenth rib. An 18 ga 6-inch spinal needle (Becton Dickinson, Franklin Lakes, NJ) that was preloaded with porcine pancreatic islets (30,000 IEQ/kg; total volume of 2.0 ml) was inserted through the skin incision into the epaxial musculature to a distance of 10 cm deep. 0.5 ml of the islet suspension was instilled, and the needle was withdrawn in 1.5 cm increments such that four total injections were made, each 2.5 cm from the previous injection site. The needle was withdrawn from the site of insertion, and the skin was sealed with tissue glue (Vetbond Tissue Adhesive™, 3M, Minneapolis, MN). Glucose tolerance tests were performed starting 3 weeks after cell implantation and were repeated at 3-5 week intervals for 28 weeks post-transplantation. An 18 ga intravenous catheter was placed in a cephalic vein. At time 0, sterile 50% glucose in water (500 mg/ml; total dose of 500 mg/kg) was given intravenously over 1-2 minutes. A 1 ml blood sample was collected prior to intravenous administration of glucose and 5, 10, 20, 60, 90, and 120 minutes after the instillation of glucose. A drop of blood was tested for glucose concentration using a glucometer (AlphaTrak, Abbott, Chicago, IL), and the remainder of the blood was placed in a tube containing EDTA. Tubes were placed on ice, and the plasma was separated by cold centrifugation at 1100×g for 10 minutes (Sorvall, ThermoScientific, Waltham, MA). Plasma was stored at −80° C. until it was tested for concentrations of C-peptide.

Western blot. Protein samples for western blotting were isolated from murine or porcine islets via homogenization with lysis buffer (#9803, CST, USA). The samples were boiled in laemmli buffer (#161-0737, BioRad, USA) for 5 minutes and were resolved on a 4-12% gradient SDS-PAGE gel and blotted to PVDF membrane. Following an overnight incubation with primary antibodies against IDO (1:1000; #86630, CST, USA) and beta-actin (1:1000, #NB600-503, Novus Biologicals, USA), detection was performed using HRP-conjugated IgG. Bands were visualized using an Azure 300 chemiluminescent imaging system (Azure Biosystems, USA).

Statistical analysis. Statistical analysis was performed using GraphPad Prism. One- and two-sided unpaired and paired t tests and one- and two-way ANOVA with Tukey's or Dunnett's tests were used for datasets with a normal distribution. P<0.05 was considered statistically significant. Data are shown as means±SEM unless otherwise noted. The sample size, n, indicates the total number of biological replicates.

### Results:

PIDO Retains Structural and Functional Characteristics of its Constituent Domains and does not Alter Islet Function

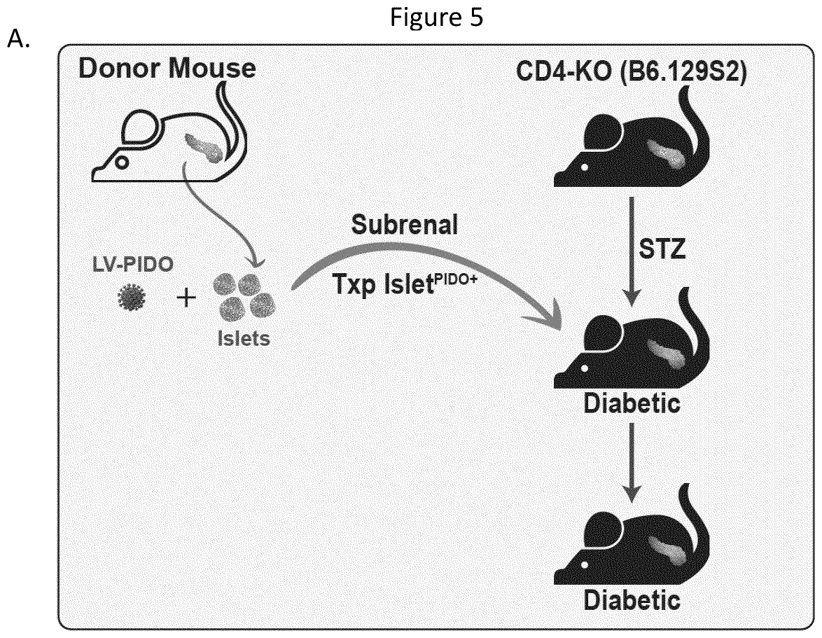

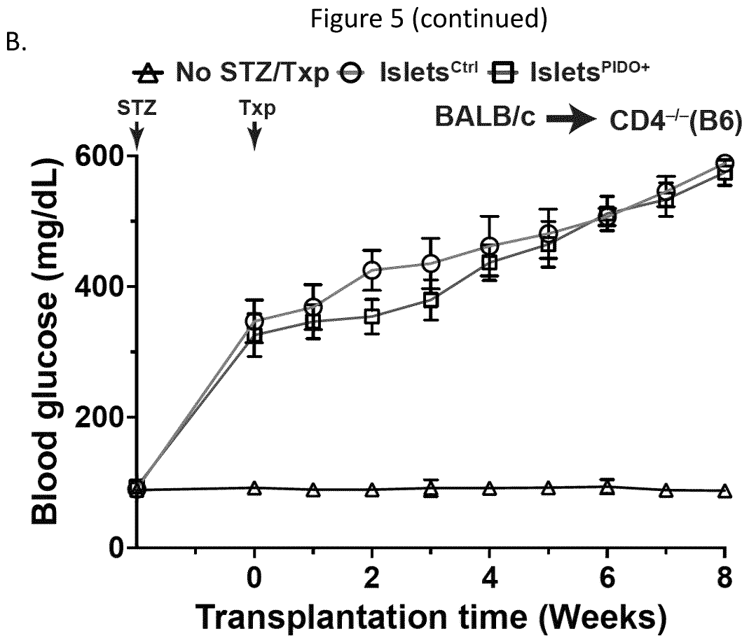

We created a synthetic gene containing sequences encoding the full-length mouse PD-L1 protein and full-length human IDO1 protein separated by a 3×GGGS linker. This synthetic gene was subcloned in-frame with the PD-L1 membrane localization signal in the pLV-EXP/CMV-EGFP lentiviral vector (FIG. 5). The resulting PIDO cDNA encodes a single polypeptide chain of 708 amino acids with a predicted non-glycosylated molecular weight of about 80 kDa (FIG. 1B). The in-silico 3D structure of PIDO was predicted and constructed using I-TASSER and webserver Phyre2 (29, 30)(FIG. 1C). The expression vector was packaged in lentiviral particles.

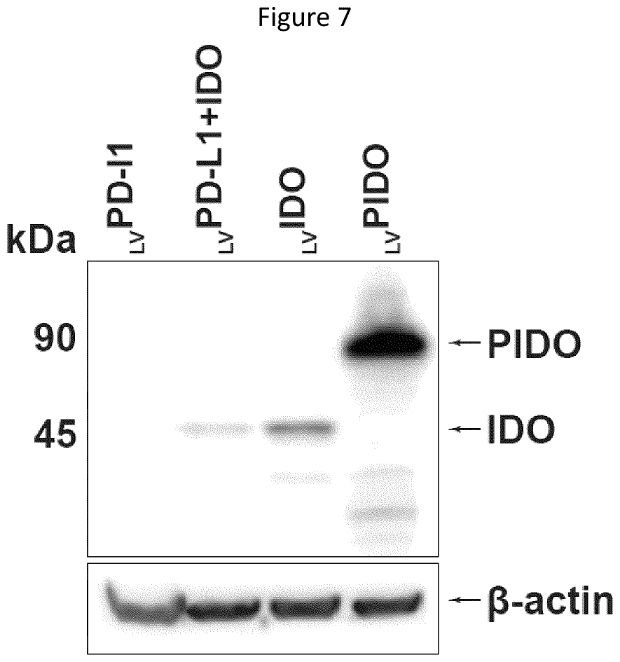

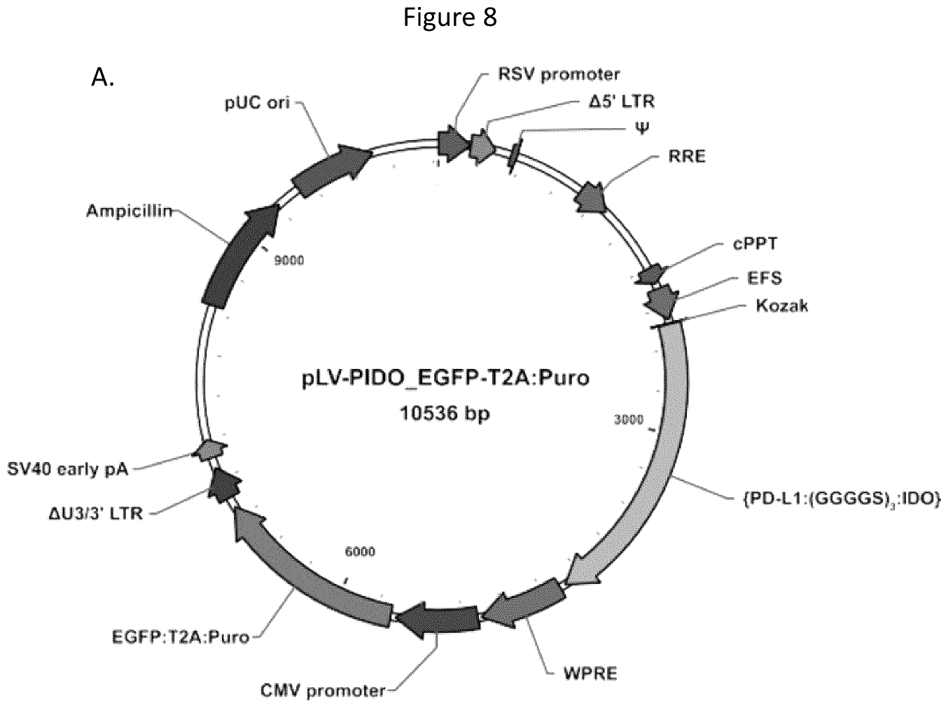

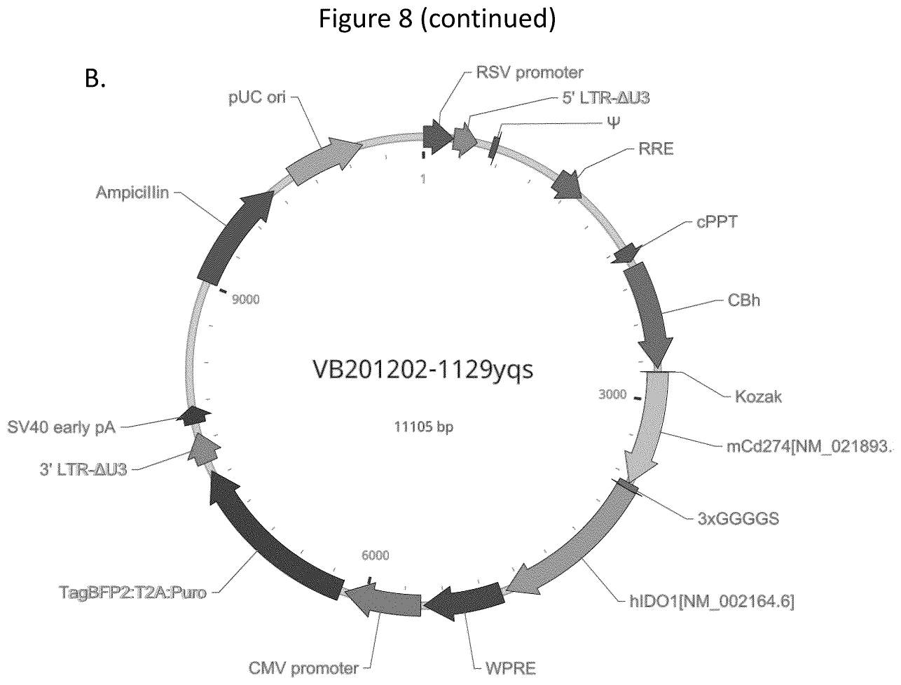

Next, we engineered A375 human melanoma cells and C57BL6/J mouse islets to express PIDO via transduction with the lentiviral particles. The expression, sub-cellular localization, and biological activity of PIDO fusion protein was verified by immunofluorescence staining, flow cytometry, western blot, and ELISA. We detected robust expression of PD-L1, IDO, and the PIDO fusion protein in mouse islets via fluorescent protein tags (FIG. 1D). To investigate the sub-cellular localization of the chimeric PIDO protein, we assessed surface expression of the PD-L1 component in dispersed islet cells by flow cytometry. Our data show that almost twice as many PIDO-expressing mouse islet cells displayed surface PD-L1 expression compared to islet cells that express PD-L1 alone (65% vs 24%), suggesting that the PIDO fusion protein allows for a higher cell surface density of PD-L1 than that afforded by ectopic expression of PD-L1 on its own (FIG. 1E). Denaturing immunoblotting performed on IDO- or PIDO-expressing mouse or pig islets showed that the fusion protein was highly expressed and migrated at a molecular weight of about 90 kDa (FIG. 1F). Our data also show that, when normalized to input protein, the abundance of the PIDO fusion protein was significantly higher than the abundance of IDO expressed alone or co-expressed with PD-L1 (FIG. 7). Together, these data suggest that PIDO-expressing islets display PD-L1 on the membrane and express IDO in the cytoplasm tethered to the C-terminus of cytoplasmic tail of PD-L1, as depicted schematically in FIG. 1G.

The activity of IDO was assessed via detection of extracellular kynurenine produced from its catalysis of tryptophan present in the culture media. As shown in FIG. 1H, kynurenine levels increased significantly in the conditioned media of both IDO- and PIDO-expressing islets, comparable the levels in the media of IFNγ-treated mesenchymal stromal cells (positive control). Interestingly, mouse islets that were dual transduced to co-express PD-L1 and IDO as separate proteins displayed lower IDO activity, as demonstrated by lower kynurenine levels in the conditioned media of these islets. This suggests that the effect of co-expression of PD-L1 and IDO is not equivalent to that of the PIDO fusion protein.

Islet β-cells are known to augment their surface expression of PD-L1 during the development of insulitis (31), potentially as a defense mechanism against autoreactive T cells. This increase in PD-L1 expression may initiate stress pathways in β-cells. Further, IDO is not naturally expressed in islets and the effects of IDO-driven tryptophan depletion and kynurenine production on β-cell function are undefined. Thus, to understand the effect of increased PD-L1 surface expression and ectopic IDO catabolic activity on these cells, we cultured islets expressing PD-L1, IDO, or PIDO for 48 hours and then subjected them to glucose-stimulated insulin secretion (GSIS) assays. The GSIS data showed no difference in insulin secretion as a function of transgene expression (FIG. 1I).

Together, these data show that the PIDO fusion protein is more stable than its protein constituents, that it is expressed robustly on the cell surface, that its IDO component retains its catalytic activity in the context of the fusion protein, and that constitutive expression of PIDO does not interfere with islet GSIS.

### PIDO-Expressing Islet Allografts Reverse Hyperglycemia in Diabetic Mice

To assess the potential of PIDO-expressing allogeneic islets for use in transplantation therapies, we transplanted ˜450 handpicked and size-matched lentivirus-transduced C57BL/6 mouse islets under the left kidney capsule of BALB/c mice that were previously rendered diabetic by streptozotocin (STZ) injection, which depletes endogenous islets (FIG. 2A). Three mice that were transplanted with islets that had been transduced with control lentivirus died spontaneously, one at 12 weeks and two others at 24 weeks, likely due to their diabetes. At 20 weeks post-transplantation, the PIDO* islet allografts were detected under the kidney capsule and stained positive for insulin (FIG. 2B). To understand whether expression of PD-L1, IDO, or both of these proteins is sufficient to reverse diabetes in mice, we also transduced C57BL/6 mouse islets with PD-L1 alone, IDO alone, or both PD-L1 and IDO as individual proteins. As is shown in FIG. 2C, islets expressing PD-L1 and/or IDO failed to reverse the preexisting hyperglycemic diabetes in mice. While the allograft recipients transplanted with islets that co-express PD-L1 and IDO showed some initial recovery (˜3 weeks post-transplantation), they never achieved normoglycemia, and by about 5 weeks post-transplantation, their initial glycemic improvement was lost. This observation further strengthens the notion that the activity of the PIDO fusion protein is superior to the combined activities of PD-L1 and IDO. Next, we tracked the blood glucose of mice with preexisting STZ-induced diabetes that were transplanted with control or PIDO-expressing islets. In PIDO* islet transplanted mice, the blood glucose dropped to less than 200 mg/dl within three weeks (FIG. 2D) and became completely normoglycemic by 10 weeks (no difference from healthy, non-transplanted mice). These PIDO* allograft recipients remained normoglycemic for the entire duration of study with an average blood glucose concentration of 87±7 mg/dl (fasting, FIG. 2D, right) or 109±12 mg/dl (random-fed, FIG. 2D, left). We performed glucose tolerance tests 2 weeks and 10 weeks post-transplantation. Mice transplanted with PIDO* islets demonstrated improved glucose tolerance as compared to control islet transplanted mice as early as 2 weeks post-transplantation (FIG. 2E). For the 50-week observation period, only PIDO* islet transplanted mice achieved and maintained normoglycemic blood glucose levels, as the mice transplanted with control islet allografts did not show any glycemic recovery. Serum was collected from all groups of mice at 2- and 10-weeks post-transplantation and was assayed for insulin. FIG. 2F shows that the PIDO* islet transplantation groups had detectable insulin at 2 weeks (0.64±0.38 ng/ml) and by 10 weeks, their insulin levels were comparable to normoglycemic, non-transplanted mice (0.9±0.17 ng/ml).

Finally, we sought to test the effect of PIDO expression on the survival of allogeneic islets in NOD mice. Control or PIDO* allogeneic (C57BL/6J) islets were transplanted into diabetic female NOD mice, and the mice were monitored for eight weeks. As is shown in FIG. 3, control islet recipients showed variable and transient improvements in blood glucose, but eventually rejected their grafts. The mean survival time of these grafts was 8 days (n=4). In contrast, PIDO* islet-recipients (n=5) showed glycemic improvement within a week, remained normoglycemic for the duration of the study (8 weeks), and showed a reversal of preexisting autoimmune diabetes (FIG. 3B, C). The relapse (blood glucose>250 mg/dL) incidence rate was 100% in the control islet recipient group and was 20% in PIDO* islet recipient group. All recipients were presumed to be non-diabetic for the ease of data visualization (FIG. 3C).

Cumulatively, these data demonstrate that constitutive PIDO expression allows islet allografts to evade immune rejection and to reverse preexisting diabetes (i.e., both chemically induced and autoimmune diabetes) in immunocompetent mice. In addition, these data also support the hypothesis that the PIDO fusion protein possesses biochemical and functional characteristics that are distinct from those of its constituent proteins.

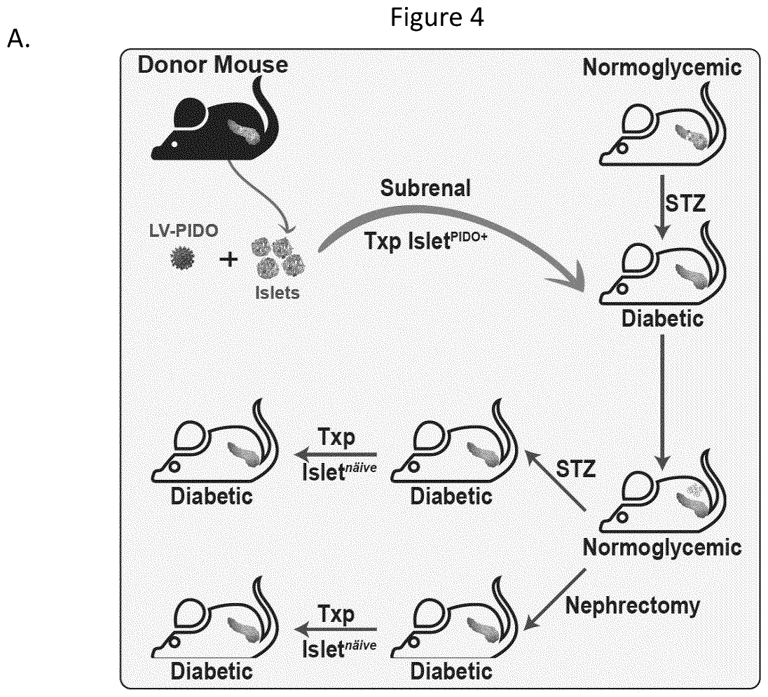

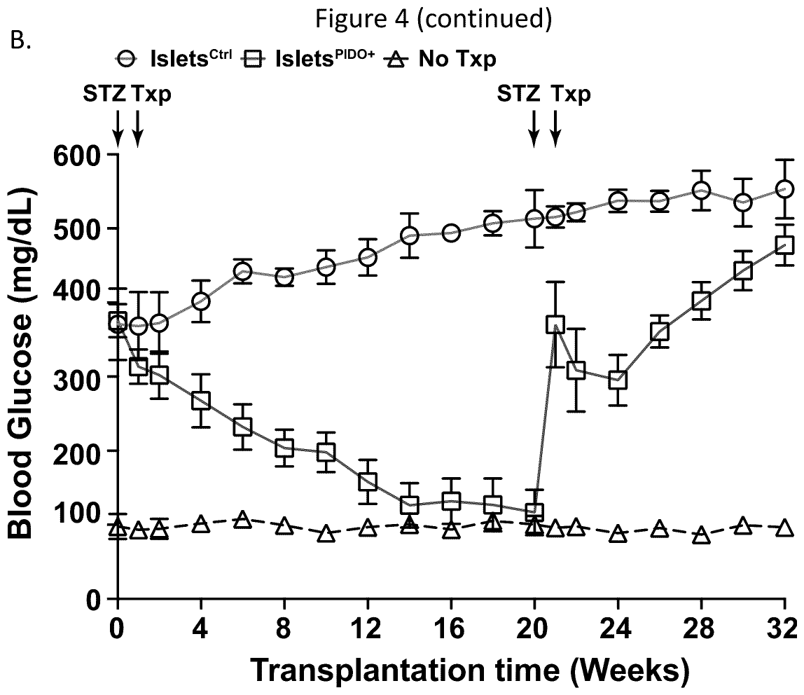

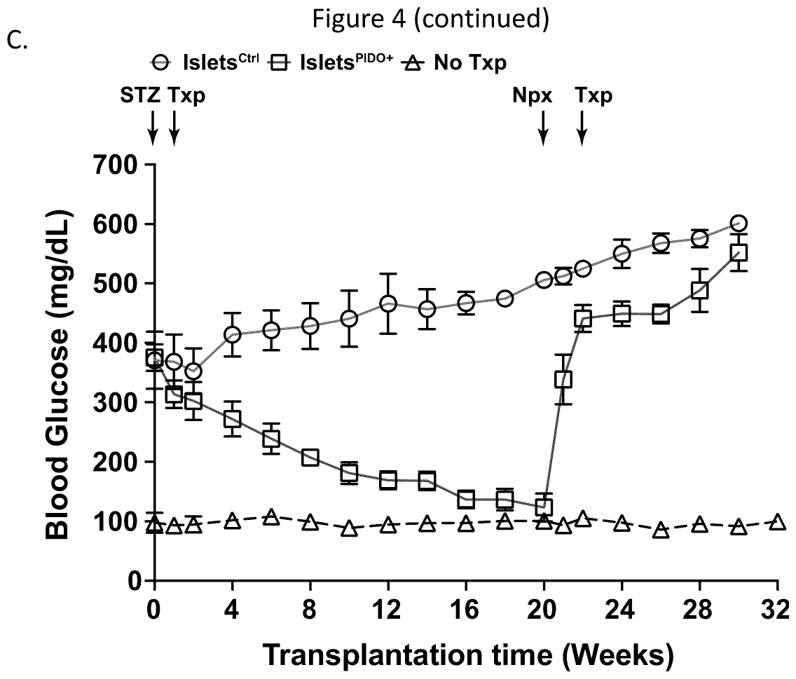

PIDO-Induced Graft Immune Evasion does not Lead to Acquired Immunologic Tolerance to Allogeneic Islets Reversal of preexisting diabetes in PIDO*islet allograft transplanted BALB/c or NOD mice is consistent with immune evasion. To test whether acquired immune tolerance of the BALB/c recipients contributes to the sustained survival of the C57BL/6 islet allografts, we destroyed/removed the PIDO* islet allografts from the BALB/c recipients via STZ treatment or nephrectomy. Thereafter, we retransplanted these mice, which were once again diabetic, with naïve C57BL/6 islets (FIG. 4A). Specifically, we injected first set of BALB/c mice (n=5) with a second dose of STZ to destroy β-cells (i.e., the PIDO* C57BL/6 islet allografts) 20 weeks post-transplantation. All recipients developed hyperglycemia within two weeks (FIG. 4B, C). Two weeks following the destruction of the primary PIDO+C57BL/6 islet allografts and re-induction of diabetes, these BALB/c mice were transplanted with a second set of naïve C57BL/6 islets under the capsule of their contralateral kidney. The naïve allografts effected only a partial and transient recovery (FIG. 4B), as these mice quickly (i.e., within 3 weeks) developed hyperglycemia, indicating a loss of the naïve allografts. Streptozotocin (STZ) is a toxic glucose analog (i.e., a DNA alkylating agent) that accumulates in islet β-cells via selective uptake by the GLUT2 glucose transporter, resulting in their destruction. While STZ is broadly used to produce a mouse model of diabetes, its efficacy in the pancreas and kidney capsule may not be equivalent due to inherent differences in the vascularization of these tissues. Thus, we hypothesized that the partial and transient glycemic recovery produced by the naïve islets could be attributed to an incomplete effect of STZ on islets under the kidney capsule. Therefore, we also tested for the existence of acquired tolerance using an independent metric. In a second set of allograft recipients (n=5), we removed the host kidney containing the PIDO*islets. These recipients developed hyperglycemia swiftly (within 1 week). Two weeks post-nephrectomy, we transplanted these BALB/c mice with naïve C57BL/6 islet allografts under the contralateral kidney capsule. All recipients became hyperglycemic within one week (FIG. 4C). Thus, the secondary naïve C57BL/6 allografts failed to reverse diabetes. These data demonstrate that the mice that had initially received PIDO*islets and were “cured” did not acquire immunologic tolerance to the allogeneic islets. Instead, the allograft tolerance achieved via PIDO expression must be mediated by immune evasion.

### PIDO-Mediated Immune Evasion Requires Host CD4 T-Cell Competence

It has been established that alloreactive tissue rejection is primarily mediated by CD8⁺ T cells (32) whereas allo-tolerance is mediated by host CD4⁺ T cells with Treg competency (33). To determine if the tolerogenic host cells that mediate PIDO-induced immune evasion are CD4⁺ T cells, we tested the therapeutic efficacy of PIDO⁺ islet allografts in CD4^{mlMak} (CD4^{-/-}) recipients that had been rendered diabetic by STZ treatment (FIG. 5A). The PIDO* islet allografts were rapidly rejected in these CD4^{-/-} mice (FIG. 5B), indicating that the responsible tolerogenic host cells are indeed CD4⁺ T-cells.

### PIDO-Expressing Porcine Pancreatic Islets are Immune Evasive in Xenogeneic Murine and Canine Recipients

While the use of gene editing methods has improved tolerance to porcine xenografts (34), immunosuppression remains necessary to prevent immune rejection of islet xenografts in non-human primates (35, 36). In view of the successful reversal of diabetes by PIDO* allografts in our murine model of allotransplantation, we next wanted to test the ability of PIDO to induce cross-species xenogeneic islet tolerance. We, therefore, created two islet xenotransplant models: a porcine-to-murine model and a porcine-to-canine model (FIG. 6A). In both models, in vitro matured juvenile porcine islets were engineered to express PIDO and were transplanted either under the kidney capsule (porcine-to-murine) or into the epaxial muscle (porcine-to-canine).

We detected porcine insulin in recipient immunocompetent hyperglycemic C57BL/6 mice up to 16 weeks post transplantation (FIG. 6B). The data show that naïve pig islet xenografts are quickly rejected and that only PIDO* pig islets survive and remain functional in the diabetic mice. However, the impact of these xenografts on clinical diabetes could not be tested using this model because porcine insulin is not compatible with the rodent insulin receptor and is, therefore, unable to regulate glucose homeostasis in mice and rats (37).

However, porcine insulin is indistinguishable from canine insulin. Thus, we also transplanted PIDO-expressing porcine islets into a normoglycemic, immunocompetent, non-diabetic beagle dog. Specifically, we tested whether a muscle implant of PIDO* porcine islets would preserve glucose homeostasis and the normal response to glucose challenge. Notably, a previous report has shown that naïve pig islets lose function quickly in diabetic canine recipients (38). The C-peptide (connecting peptide) is a short polypeptide that connects insulin's A- and B-chains in the proinsulin molecule. C-peptide is a marker of insulin secretion, as it is cleaved during mature insulin production and secreted along with insulin. Thus, to determine the effect of the muscle implant on insulin secretion, we measured the porcine C-peptide in canine plasma. (Note: This is feasible because the porcine C-peptide has negligible cross-reactivity with the canine C-peptide.) We first performed an intravenous glucose tolerance test (ivGTT) to invoke a response in the euglycemic dog, which would otherwise not recruit the porcine islets due to its fully competent endogenous islet mass. We then detected porcine C-peptide for 20 weeks in dog plasma in response to the glucose stimulus (FIG. 6C). These data strongly suggests that the porcine islet xenograft survived.

Interestingly, we also observed a progressive decline in C-peptide response to GTT over time. This decline, however, cannot be attributed solely to a loss of xenograft due to immune rejection as the duration of detectable graft function extended well beyond the known period of immune rejection in the canine recipients (38).

### Discussion:

Allogeneic pancreatic islet transplantation is a potentially life-saving therapy for poorly controlled diabetes mellitus. However, adverse effects of systemic chronic pharmacological immunosuppression significantly limit the benefits and consequently the adaptation of this therapy (4). Strategies for enabling pharmacopeia-free durable allogeneic islet immune evasion are needed (39).

Knowledge gained from the field of cancer immunotherapy, which is focused on eliminating immune evasion, provides insights as to how to achieve allogeneic tissue tolerance. Malignancies often exploit several immunosuppressive pathways to evade an immune response. The PD-1:PD-L1 (40) and IDO (41) pathways are both implicated in such microenvironments and have been recognized as important immune checkpoints. Thus, oncology researchers view these pathways as potential therapeutic targets and have attempted to block them and have shown the biological potency of immunological escape for select malignant disorders with high mutation burden. For example, researchers recently tested the utility of constitutive expression of PD-L1 by human islet-like organoids as a means to evade xenorejection in mice (27). In analogous work, PD-L1-expressing islets on a microgel/biomaterial platform bypassed the need for genetic modification of the graft cell/tissue (24, 42) by transient expression of PD-L1. However, this failed to provide sustained protection of islets against alloreactive responses and contemporaneous pharmacological immunosuppression was necessary. This work demonstrated the need for an approach that can provide specific, localized, and durable immune evasion while circumventing the need for immunosuppression.

In the present study, we generated a novel chimeric fusion protein comprising PD-L1 and IDO. By harnessing the immune evasive potential of both the PD-1:PD-L1 and IDO pathways, we sought to modulate the alloreactive immune response against pancreatic islet allografts in murine recipients. PD-L1 and IDO have not been used together before as an immune blockade therapeutic. The observations made herein suggest that tethering IDO to the cytoplasmic tail of PD-L1 produces beneficial gain-of-function properties that are not achieved via simultaneous independent expression of these proteins. We observed that both immortalized cell lines and primary islet cells that express PIDO displayed PD-L1 on the surface and enzymatically active IDO in the cytoplasm. The PIDO fusion protein not only retained the biological functions of both constituent proteins, but it also granted its constituent proteins enhanced stability. We tested the efficacy of PIDO in immunoprotection of allografts by generating PIDO-expressing islets which, after transplantation, reversed preexisting diabetes in STZ-diabetic mice and established sustained euglycemia for more than 50 weeks without immunosuppression.

In agreement with some previous studies, we observed that stable expression of PD-L1 or IDO individually did not meaningfully improve graft survival. Interestingly, we also observed that co-expression of PD-L1 and IDO only delayed the immune rejection of islet allografts temporarily. These observations reveal that while PD-L1 and IDO are insufficient individually, the PIDO fusion protein can establish and maintain an immune evasive microenvironment that protects allografts from rejection long term.

Non-specific, off-target effects are always a concern with ectopic expression of immunomodulatory proteins. However, we did not observe any effect of PIDO expression on features of bona fide mature islet β-cells such as robust dynamic function or diabetes reversal upon transplantation. Similarly, the absence of meaningful cellular proliferation (data not shown) in homeostatic conditions in mature, terminally differentiated islet β-cells (43) remained uninfluenced by PIDO expression.

Type I (i.e., autoimmune) diabetes will be the primary application for any treatment that enables immune evasion for islet allografts. This prompted us to determine the therapeutic effects of PIDO-expressing islets in the NOD mouse T1D model. In this model, we showed that blood glucose levels of PIDO* islet recipients decreased to ˜230 mg/dl (as compared to −450 mg/dl in the control diabetic NOD mice) within three weeks after transplantation and continued to improve further. Death of mice in the control islet group also suggested that there would be significant difference in survival. These data indicate that PIDO-mediated immune evasion protects islet allografts from autoimmune destruction and consequently reverses diabetes in NOD mice. Previous studies (44, 45) have revealed that induced expansion or differentiation of Tregs prolongs survival in diabetic NOD mice. Since both the PD-L1 and IDO pathways converge on Treg induction, we hypothesized that the impact of PIDO on allograft survival may be related to host CD4 T-cell competency. Our observation that PIDO expression by allogeneic islets elicits an endogenous CD4-dependent immune evasive response is consistent with the central role of host acquired T-cell drivers of tolerance. Among various traditional and novel tolerogenic approaches to prevent graft rejection, allo- and autoreactive T cell suppression via Treg cell therapy has shown feasibility, tolerability, and potential efficacy in transplantation settings (46-49). However, these approaches (including Treg enhancing drugs and antigen-specific Treg cell therapies) have demonstrated only modest and limited efficacy in T1D and transplant rejection in clinical settings (50, 51). Islet-restricted, constitutive PIDO expression and its attendant host CD4-dependent immune evasion may address the shortcomings of Treg adoptive cell therapies via continuous in vivo solicitation of endogenous regulatory CD4⁺ cells.

We observed that PIDO expression led to significantly improved, immunosuppression-free, long-term engraftment of allogeneic islets and consequently, reversal of diabetes and maintenance of euglycemia for more than 50 weeks in murine recipients. This led us to hypothesize that PIDO was leading to the development of acquired tolerance for alloantigens in the murine hosts. However, using two different models of rechallenge, we determined that long-term (20-weeks) localized PIDO expression did not establish acquired memory tolerance, as the hosts rejected naïve islet allografts promptly (˜3 weeks) after re-transplantation. These results demonstrate that PIDO must be constitutively expressed by islets to confer immune evasive qualities.

Success in xenogeneic tissue transplantation has remained elusive. Rare and moderate improvements have been reported among the few attempts that have been made towards achieving xenograft tolerance (35, 37, 38), and there are virtually no published reports of immunosuppressant-free survival of xenografts in immune sufficient mammalian recipients. To test the efficacy of PIDO in this application, we transplanted porcine islet xenografts into immunocompetent mice and dogs. We observed a substantial prolongation of xenograft survival in both the murine (˜16 weeks) and canine (˜20 weeks) recipients. However, there are several important limitations to these xenograft survival experiments. First, our results were obtained from normoglycemic recipients, which prevented us from testing the ability of the islet xenografts to reverse pathological hyperglycemia. Second, to glean as much information possible from as few experimental canines as possible, we studied porcine islet xenograft in only a singular canine recipient. While the results of these experiments are promising, differences in functional integration of ectopic islet grafts are unknown, especially under metabolic stress conditions. Thus, further in-depth studies of more animals and different models is warranted to better understand the PIDO-mediated improvement in xenograft islet survival and function. Lastly, the murine model of diabetes that we utilized for xenogeneic transplant is representative of drug induced (STZ) islet insufficiency and secondary diabetes mellitus. Though this model system mirrors clinical diabetes caused by non-immune pancreatic insufficiency or pancreatectomy, it does not reflect the pathology of autoimmune islet destruction typically seen in type I diabetes. However, our data generated in diabetic NOD mice suggest that PIDO enables immune evasion in the context of autoimmune diabetes as well.

Taken together, our data support the use of PIDO as a novel immune evasive blockade therapeutic that effectively prevents islet allograft rejection in immunocompetent recipients and successfully circumvents the need for immunosuppressive therapy. While the mechanism by which PIDO establishes non-memory immune evasion remains to be elucidated, we hypothesize that it involves evasion of both innate and adaptive immune responses. In conclusion, expression of the PIDO fusion protein may allow off-the-shelf islet transplants to be used as a standard therapy for treating poorly controlled insulin-dependent diabetes.

### REFERENCES

1. Justiz Vaillant A A, Misra S, Fitzgerald B M. Acute Transplantation Rejection. In. StatPearls. Treasure Island (F L), 2021.
2. Molnar A O, Fergusson D, Tsampalieros A K, Bennett A, Fergusson N, Ramsay T et al. Generic immunosuppression in solid organ transplantation: systematic review and meta-analysis. BMJ 2015; 350:h3163.
3. Kawase T, Matsuo K, Kashiwase K, Inoko H, Saji H, Ogawa S et al. HLA mismatch combinations associated with decreased risk of relapse: implications for the molecular mechanism. Blood 2009; 113(12):2851-2858.
4. Wood K J, Goto R. Mechanisms of rejection: current perspectives. Transplantation 2012; 93(1):1-10.
5. Calne R. Clinical transplantation: current problems, possible solutions. Philos Trans R Soc Lond B Biol Sci 2005; 360(1461):1797-1801.
6. Barton F B, Rickels M R, Alejandro R, Hering B J, Wease S, Naziruddin B et al. Improvement in outcomes of clinical islet transplantation: 1999-2010. Diabetes Care 2012; 35(7):1436-1445.
7. Robertson R P. Islet transplantation as a treatment for diabetes—a work in progress. N Engl J Med 2004; 350(7):694-705.
8. Rother K I, Harlan D M. Challenges facing islet transplantation for the treatment of type 1 diabetes mellitus. J Clin Invest 2004; 114(7):877-883.
9. Shapiro A M, Ricordi C, Hering B J, Auchincloss H, Lindblad R, Robertson R P et al. International trial of the Edmonton protocol for islet transplantation. N Engl J Med 2006; 355(13):1318-1330.
10. Bluestone J A, Anderson M. Tolerance in the Age of Immunotherapy. N Engl J Med 2020; 383(12):1156-1166.
11. Jiang Y, Chen M, Nie H, Yuan Y. PD-1 and PD-L1 in cancer immunotherapy: clinical implications and future considerations. Hum Vaccin Immunother 2019; 15(5):1111-1122.
12. Keir M E, Butte M J, Freeman G J, Sharpe A H. PD-1 and its ligands in tolerance and immunity. Annu Rev Immunol 2008; 26:677-704.
13. Colli M L, Hill J L E, Marroqui L, Chaffey J, Dos Santos R S, Leete P et al. PDL1 is expressed in the islets of people with type 1 diabetes and is up-regulated by interferons-alpha and-gamma via IRF1 induction. EBioMedicine 2018; 36:367-375.
14. Ishida M, Iwai Y, Tanaka Y, Okazaki T, Freeman G J, Minato N et al. Differential expression of PD-L1 and PD-L2, ligands for an inhibitory receptor PD-1, in the cells of lymphohematopoietic tissues. Immunol Lett 2002; 84(1):57-62.
15. de Filette J M K, Pen J J, Decoster L, Vissers T, Bravenboer B, Van der Auwera B J et al. Immune checkpoint inhibitors and type 1 diabetes mellitus: a case report and systematic review. Eur J Endocrinol 2019; 181(3):363-374.
16. Cai J, Wang D, Zhang G, Guo X. The Role Of PD-1/PD-L1 Axis In Treg Development And Function: Implications For Cancer Immunotherapy. Onco Targets Ther 2019; 12:8437-8445.
17. Wang J, Yoshida T, Nakaki F, Hiai H, Okazaki T, Honjo T. Establishment of NOD-Pdcdl−/− mice as an efficient animal model of type I diabetes. Proc Natl Acad Sci USA 2005; 102(33):11823-11828.
18. Munn D H, Zhou M, Attwood J T, Bondarev I, Conway S J, Marshall B et al. Prevention of allogeneic fetal rejection by tryptophan catabolism. Science 1998; 281(5380):1191-1193.
19. Munn D H, Sharma M D, Baban B, Harding H P, Zhang Y, Ron D et al. GCN2 kinase in T cells mediates proliferative arrest and anergy induction in response to indoleamine 2,3-dioxygenase. Immunity 2005; 22(5):633-642.
20. Huang L, Baban B, Johnson B A, 3rd, Mellor A L. Dendritic cells, indoleamine 2,3 dioxygenase and acquired immune privilege. Int Rev Immunol 2010; 29(2):133-155.
21. Munn D H, Mellor A L, Rossi M, Young J W. Dendritic cells have the option to express IDO-mediated suppression or not. Blood 2005; 105(6):2618.
22. Litzenburger U M, Opitz C A, Sahm F, Rauschenbach K J, Trump S, Winter M et al. Constitutive IDO expression in human cancer is sustained by an autocrine signaling loop involving IL-6, STAT3 and the AHR. Oncotarget 2014; 5(4):1038-1051.
23. McGrath M M, Najafian N. The role of coinhibitory signaling pathways in transplantation and tolerance. Front Immunol 2012; 3:47.
24. Batra L, Shrestha P, Zhao H, Woodward K B, Togay A, Tan M et al. Localized Immunomodulation with PD-L1 Results in Sustained Survival and Function of Allogeneic Islets without Chronic Immunosuppression. J Immunol 2020.
25. Liu H, Liu L, Fletcher B S, Visner G A. Sleeping Beauty-based gene therapy with indoleamine 2,3-dioxygenase inhibits lung allograft fibrosis. FASEB J 2006; 20(13):2384-2386.
26. Jalili R B, Forouzandeh F, Rezakhanlou A M, Hartwell R, Medina A, Warnock G L et al. Local expression of indoleamine 2,3 dioxygenase in syngeneic fibroblasts significantly prolongs survival of an engineered three-dimensional islet allograft. Diabetes 2010; 59(9):2219-2227.
27. Yoshihara E, O'Connor C, Gasser E, Wei Z, Oh T G, Tseng T W et al. Immune-evasive human islet-like organoids ameliorate diabetes. Nature 2020; 586(7830):606-611.
28. Li R, Lee J, Kim M S, Liu V, Moulik M, Li H et al. PD-L1-driven tolerance protects neurogenin3-induced islet neogenesis to reverse established type 1 diabetes in NOD mice. Diabetes 2015; 64(2):529-540.
29. Kelley L A, Mezulis S, Yates C M, Wass M N, Sternberg M J. The Phyre2 web portal for protein modeling, prediction and analysis. Nat Protoc 2015; 10(6):845-858.
30. Yang J, Zhang Y. Protein Structure and Function Prediction Using I-TASSER. Curr Protoc Bioinformatics 2015; 52:5 8 1-5 8 15.
31. Osum K C, Burrack A L, Martinov T, Sahli N L, Mitchell J S, Tucker C G et al. Interferon-gamma drives programmed death-ligand 1 expression on islet beta cells to limit T cell function during autoimmune diabetes. Sci Rep 2018; 8(1):8295.
32. Marino J, Paster J, Benichou G. Allorecognition by T Lymphocytes and Allograft Rejection. Front Immunol 2016; 7:582.
33. Walsh P T, Taylor D K, Turka L A. Tregs and transplantation tolerance. J Clin Invest 2004; 114(10):1398-1403.
34. Cowan P J. The use of CRISPR/Cas associated technologies for cell transplant applications. Curr Opin Organ Transplant 2016; 21(5):461-466.
35. Hering B J, Wijkstrom M, Graham M L, Hardstedt M, Aasheim T C, Jie T et al. Prolonged diabetes reversal after intraportal xenotransplantation of wild-type porcine islets in immunosuppressed nonhuman primates. Nat Med 2006; 12(3):301-303.
36. Shin J S, Kim J M, Kim J S, Min B H, Kim Y H, Kim H J et al. Long-term control of diabetes in immunosuppressed nonhuman primates (NHP) by the transplantation of adult porcine islets. Am J Transplant 2015; 15(11):2837-2850.
37. Pepper A R, Gall C, Mazzuca D M, Melling C W, White D J. Diabetic rats and mice are resistant to porcine and human insulin: flawed experimental models for testing islet xenografts. Xenotransplantation 2009; 16(6):502-510.
38. Kin T, Iwata H, Aomatsu Y, Ohyama T, Kanehiro H, Hisanaga M et al. Xenotransplantation of pig islets in diabetic dogs with use of a microcapsule composed of agarose and polystyrene sulfonic acid mixed gel. Pancreas 2002; 25(1):94-100.
39. Melton D. The promise of stem cell-derived islet replacement therapy. Diabetologia 2021; 64(5):1030-1036.
40. Dermani F K, Samadi P, Rahmani G, Kohlan A K, Najafi R. PD-1/PD-L1 immune checkpoint: Potential target for cancer therapy. J Cell Physiol 2019; 234(2):1313-1325.
41. Zhai L, Bell A, Ladomersky E, Lauing K L, Bollu L, Sosman J A et al. Immunosuppressive IDO in Cancer: Mechanisms of Action, Animal Models, and Targeting Strategies. Front Immunol 2020; 11:1185.
42. Coronel M M, Martin K E, Hunckler M D, Barber G, O'Neill E B, Medina J D et al. Immunotherapy via PD-L1-presenting biomaterials leads to long-term islet graft survival. Sci Adv 2020; 6(35):eaba5573.
43. Paul P K, Rabaglia M E, Wang C Y, Stapleton D S, Leng N, Kendziorski C et al. Histone chaperone ASF1B promotes human beta-cell proliferation via recruitment of histone H3.3. Cell Cycle 2016; 15(23):3191-3202.
44. Lin J R, Huang S H, Wu C H, Chen Y W, Hong Z J, Cheng C P et al. Valproic Acid Suppresses Autoimmune Recurrence and Allograft Rejection in Islet Transplantation through Induction of the Differentiation of Regulatory T Cells and Can Be Used in Cell Therapy for Type 1 Diabetes. Pharmaceuticals (Basel) 2021; 14(5).
45. Shi Q, Lees J R, Scott D W, Farber D L, Bartlett S T. Endogenous expansion of regulatory T cells leads to long-term islet graft survival in diabetic NOD mice. Am J Transplant 2012; 12(5):1124-1132.
46. Bruder D, Westendorf A M, Hansen W, Prettin S, Gruber A D, Qian Y et al. On the edge of autoimmunity: T-cell stimulation by steady-state dendritic cells prevents autoimmune diabetes. Diabetes 2005; 54(12):3395-3401.
47. Mukhopadhaya A, Hanafusa T, Jarchum I, Chen Y G, Iwai Y, Serreze D V et al. Selective delivery of beta cell antigen to dendritic cells in vivo leads to deletion and tolerance of autoreactive CD8+ T cells in NOD mice. Proc Natl Acad Sci USA 2008; 105(17):6374-6379.
48. Ryba-Stanislawowska M, Sakowska J, Zielinski M, Lawrynowicz U, Trzonkowski P. Regulatory T cells: the future of autoimmune disease treatment. Expert Rev Clin Immunol 2019; 15(7):777-789.
49. Sharabi A, Tsokos M G, Ding Y, Malek T R, Klatzmann D, Tsokos G C. Regulatory T cells in the treatment of disease. Nat Rev Drug Discov 2018; 17(11):823-844.
50. Bluestone J A, Buckner J H, Fitch M, Gitelman S E, Gupta S, Hellerstein M K et al. Type 1 diabetes immunotherapy using polyclonal regulatory T cells. Sci Transl Med 2015; 7(315):315ra189.
51. MacDonald K G, Hoeppli R E, Huang Q, Gillies J, Luciani D S, Orban P C et al. Alloantigen-specific regulatory T cells generated with a chimeric antigen receptor. J Clin Invest 2016; 126(4):1413-1424.
52. Vanderschelden R, Sathialingam M, Alexander M, Lakey J R T. Cost and Scalability Analysis of Porcine Islet Isolation for Islet Transplantation: Comparison of Juvenile, Neonatal and Adult Pigs. Cell Transplant 2019; 28(7):967-972.
53. de Souza A H, Santos L R B, Roma L P, Bensellam M, Carpinelli A R, Jonas J C. NADPH oxidase-2 does not contribute to beta-cell glucotoxicity in cultured pancreatic islets from C57BL/6J mice. Mol Cell Endocrinol 2017; 439:354-362.

1. A fusion protein comprising from N-terminus to C-terminus:
a) a programmed death ligand-1 (PD-L1) peptide comprising at least a portion of the extracellular domain of a PD-L1 protein,
b) a transmembrane domain, and
c) an indolamine 2,3-dioxygenase (IDO) peptide comprising at least a portion of an IDO protein;
optionally wherein the PD-L1 peptide is capable of binding to PD-1 and the IDO peptide is catalytically active.

2. The fusion protein of claim 1, wherein the PD-L1 peptide has at least 95% identity to SEQ ID NO:3 or SEQ ID NO:7.

3. The fusion protein of claim 1, wherein the PD-L1 peptide further comprises a PD-L1 signal peptide.

4. The fusion protein of claim 3, wherein the PD-L1 signal peptide is SEQ ID NO:4 or SEQ ID NO:8.

5. The fusion protein of claim 1, wherein the transmembrane domain comprises at least a portion of the transmembrane domain of a PD-L1 protein.

6. The fusion protein of claim 5, wherein the transmembrane domain has at least 95% identity to SEQ ID NO:5 or SEQ ID NO:9.

7. The fusion protein of claim 1, wherein the IDO peptide has at least 95% identity to SEQ ID NO:10.

8. The fusion protein of claim 1, wherein the transmembrane domain is linked to the IDO peptide by a linker peptide.

9. (canceled)

10. (canceled)

11. The fusion protein of claim 1, wherein the fusion protein comprises SEQ ID NO:1, SEQ ID NO:14, SEQ ID NO:17, or SEQ ID NO:20.

12. A nucleic acid construct comprising a polynucleotide encoding the fusion protein of claim 1 operably linked to a promoter.

13. The nucleic acid construct of claim 12, wherein the promoter is an elongation factor 1α short (EFS) promoter or a hybrid CMV enhancer/chicken β-actin (CBA) promoter.

14. The nucleic acid construct of claim 12, wherein the nucleic acid construct is a viral vector.

15. (canceled)

16. A cell comprising the nucleic acid construct of claim 12.

17. The cell of claim 16, wherein the cell expresses a fusion protein comprising from N-terminus to C-terminus:
a) a programmed death ligand-1 (PD-L1) peptide comprising at least a portion of the extracellular domain of a PD-L1 protein,
b) a transmembrane domain, and
c) an indolamine 2,3-dioxygenase (IDO) peptide comprising at least a portion of an IDO protein.

18. (canceled)

19. The cell of claim 17, wherein the PD-L1 peptide is localized in the extracellular space and the IDO peptide is localized in the cytoplasm of the cell.

20. The cell of claim 16, wherein the cell is an islet, induced pluripotent stem cell, embryonic stem cell, retinal pigment epithelial cell, dopaminergic neuron, or cardiomyocyte.

21. A method of transplanting the cell of claim 16 into a subject.

22. The method of claim 21, wherein the cell is from an allogenic source or a xenogeneic source.

23. (canceled)

24. (canceled)

25. (canceled)

26. The method of claim 21, wherein the transplanted cell performs its native function, is tolerated by the immune system in the absence of immunosuppression, or has prolonged survival relative to a transplanted control cell lacking the nucleic acid construct.

27. (canceled)

28. (canceled)

29. The method of claim 21, wherein the subject is diabetic, the cell is an islet, and the cell produces insulin post-transplantation.

30. (canceled)

31. (canceled)

32. The method of claim 29, wherein the subject demonstrates improved glucose tolerance post-transplantation as compared to pre-transplantation, becomes normoglycemic post-transplantation, or remains normoglycemic for at least 50 weeks post-transplantation.

33. (canceled)

34. (canceled)


SEQUENCE LISTING (24 sequences)

SEQ ID NO: 1 - protein, 709 aa, Artificial Sequence
  Synthetic - PIDO fusion protein comprising mouse PD-L1
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHWVLLGSILLFLIVVSTVLLFLRKQVRMLDVEKCGVEDTSSKNRNDTQFEETSGGGGSGGGGSGGGGSMAHAMENSWTISKEYHIDEEVGFALPNPQENLPDFYNDWMFIAKHLPDLIESGQLRERVEKLNMLSIDHLTDHKSQRLARLVLGCITMAYVWGKGHGDVRKVLPRNIAVPYCQLSKKLELPPILVYADCVLANWKKKDPNKPLTYENMDVLFSFRDGDCSKGFFLVSLLVEIAAASAIKVIPTVFKAMQMQERDTLLKALLEIASCLEKALQVFHQIHDHVNPKAFFSVLRIYLSGWKGNPQLSDGLVYEGFWEDPKEFAGGSAGQSSVFQCFDVLLGIQQTAGGGHAAQFLQDMRRYMPPAHRNFLCSLESNPSVREFVLSKGDAGLREAYDACVKALVSLRSYHLQIVTKYILIPASQQPKENKTSEDPSKLEAKGTGGTDLMNFLKTVRSTTEKSLLKEG

SEQ ID NO: 2 - protein, 290 aa, Mus musculus
  MISC_FEATURE (1)..(290): Full-length mouse PD-L1 protein
  MISC_FEATURE (121)..(125): ADYKR sequence required for PD-1 binding domain
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHWVLLGSILLFLIVVSTVLLFLRKQVRMLDVEKCGVEDTSSKNRNDTQFEET

SEQ ID NO: 3 - protein, 221 aa, Mus musculus
  MISC_FEATURE (1)..(221): Extracellular domain of mouse PD-L1 (amino acids 19-239 of SEQ ID NO 2)
FTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTH

SEQ ID NO: 4 - protein, 18 aa, Mus musculus
  MISC_FEATURE (1)..(18): Signal peptide of mouse PD-L1 (amino acids 1-18 of SEQ ID NO 2; cleaved off in mature protein)
MRIFAGIIFTACCHLLRA

SEQ ID NO: 5 - protein, 21 aa, Mus musculus
  misc_feature (1)..(21): Transmembrane domain of mouse PD-L1 (amino acids 240-260 of SEQ ID NO 2)
WVLLGSILLFLIVVSTVLLFL

SEQ ID NO: 6 - protein, 290 aa, Homo sapiens
  MISC_FEATURE (1)..(290): Full-length human PD-L1 protein
  MISC_FEATURE (121)..(125): ADYKR sequence required for PD-1 binding domain
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 7 - protein, 220 aa, Homo sapiens
  MISC_FEATURE (1)..(220): Extracellular domain of human PD-L1 (amino acids 19-238 of SEQ ID NO6)
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 8 - protein, 18 aa, Homo sapiens
  MISC_FEATURE (1)..(18): Signal peptide of human PD-L1 (amino acids 1-18 of SEQ ID NO6; cleaved off in mature protein)
MRIFAVFIFMTYWHLLNA

SEQ ID NO: 9 - protein, 21 aa, Homo sapiens
  MISC_FEATURE (1)..(21): Transmembrane domain of human PD-L1 (amino acids 239-259 of SEQ ID NO6)
THLVILGAILLCLGVALTFIF

SEQ ID NO: 10 - protein, 403 aa, Homo sapiens [sequence omitted]

SEQ ID NO: 11 - protein, 16 aa, Artificial Sequence
  Synthetic - 3X GGGS linker
SGGGGSGGGGSGGGGS

SEQ ID NO: 12 - DNA, 2130 bp, Artificial Sequence
  Synthetic - DNA encoding PIDO fusion protein comprising mouse PD-L1
atgaggatatttgctggcattatattcacagcctgctgtcacttgctacgggcgtttactatcacggctccaaaggacttgtacgtggtggagtatggcagcaacgtcacgatggagtgcagattccctgtagaacgggagctggacctgcttgcgttagtggtgtactgggaaaaggaagatgagcaagtgattcagtttgtggcaggagaggaggaccttaagcctcagcacagcaacttcagggggagagcctcgctgccaaaggaccagcttttgaagggaaatgctgcccttcagatcacagacgtcaagctgcaggacgcaggcgtttactgctgcataatcagctacggtggtgcggactacaagcgaatcacgctgaaagtcaatgccccataccgcaaaatcaaccagagaatttccgtggatccagccacttctgagcatgaactaatatgtcaggccgagggttatccagaagctgaggtaatctggacaaacagtgaccaccaacccgtgagtgggaagagaagtgtcaccacttcccggacagaggggatgcttctcaatgtgaccagcagtctgagggtcaacgccacagcgaatgatgttttctactgtacgttttggagatcacagccagggcaaaaccacacagcggagctgatcatcccagaactgcctgcaacacatcctccacagaacaggactcactgggtgcttctgggatccatcctgttgttcctcattgtagtgtccacggtcctcctcttcttgagaaaacaagtgagaatgctagatgtggagaaatgtggcgttgaagatacaagctcaaaaaaccgaaatgatacacaattcgaggagacgtctggtggcggaggctcgggcggaggtgggtcgggtggcggcggatcaatggcacacgctatggaaaactcctggacaatcagtaaagagtaccatattgatgaagaagtgggctttgctctgccaaatccacaggaaaatctacctgatttttataatgactggatgttcattgctaaacatctgcctgatctcatagagtctggccagcttcgagaaagagttgagaagttaaacatgctcagcattgatcatctcacagaccacaagtcacagcgccttgcacgtctagttctgggatgcatcaccatggcatatgtgtggggcaaaggtcatggagatgtccgtaaggtcttgccaagaaatattgctgttccttactgccaactctccaagaaactggaactgcctcctattttggtttatgcagactgtgtcttggcaaactggaagaaaaaggatcctaataagcccctgacttatgagaacatggacgttttgttctcatttcgtgatggagactgcagtaaaggattcttcctggtctctctattggtggaaatagcagctgcttctgcaatcaaagtaattcctactgtattcaaggcaatgcaaatgcaagaacgggacactttgctaaaggcgctgttggaaatagcttcttgcttggagaaagcccttcaagtgtttcaccaaatccacgatcatgtgaacccaaaagcatttttcagtgttcttcgcatatatttgtctggctggaaaggcaacccccagctatcagacggtctggtgtatgaagggttctgggaagacccaaaggagtttgcagggggcagtgcaggccaaagcagcgtctttcagtgctttgacgtcctgctgggcatccagcagactgctggtggaggacatgctgctcagttcctccaggacatgagaagatatatgccaccagctcacaggaacttcctgtgctcattagagtcaaatccctcagtccgtgagtttgtcctttcaaaaggtgatgctggcctgcgggaagcttatgacgcctgtgtgaaagctctggtctccctgaggagctaccatctgcaaatcgtgactaagtacatcctgattcctgcaagccagcagccaaaggagaataagacctctgaagacccttcaaaactggaagccaaaggaactggaggcactgatttaatgaatttcctgaagactgtaagaagtacaactgagaaatcccttttgaaggaaggttaa

SEQ ID NO: 13 - DNA, 11105 bp, Artificial Sequence
  Synthetic - Lentiviral vector encoding PIDO fusion protein comprising mouse PD-L1
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcaagtttgtacaaaaaagcaggctgccaccatgaggatatttgctggcattatattcacagcctgctgtcacttgctacgggcgtttactatcacggctccaaaggacttgtacgtggtggagtatggcagcaacgtcacgatggagtgcagattccctgtagaacgggagctggacctgcttgcgttagtggtgtactgggaaaaggaagatgagcaagtgattcagtttgtggcaggagaggaggaccttaagcctcagcacagcaacttcagggggagagcctcgctgccaaaggaccagcttttgaagggaaatgctgcccttcagatcacagacgtcaagctgcaggacgcaggcgtttactgctgcataatcagctacggtggtgcggactacaagcgaatcacgctgaaagtcaatgccccataccgcaaaatcaaccagagaatttccgtggatccagccacttctgagcatgaactaatatgtcaggccgagggttatccagaagctgaggtaatctggacaaacagtgaccaccaacccgtgagtgggaagagaagtgtcaccacttcccggacagaggggatgcttctcaatgtgaccagcagtctgagggtcaacgccacagcgaatgatgttttctactgtacgttttggagatcacagccagggcaaaaccacacagcggagctgatcatcccagaactgcctgcaacacatcctccacagaacaggactcactgggtgcttctgggatccatcctgttgttcctcattgtagtgtccacggtcctcctcttcttgagaaaacaagtgagaatgctagatgtggagaaatgtggcgttgaagatacaagctcaaaaaaccgaaatgatacacaattcgaggagacgtctggtggcggaggctcgggcggaggtgggtcgggtggcggcggatcaatggcacacgctatggaaaactcctggacaatcagtaaagagtaccatattgatgaagaagtgggctttgctctgccaaatccacaggaaaatctacctgatttttataatgactggatgttcattgctaaacatctgcctgatctcatagagtctggccagcttcgagaaagagttgagaagttaaacatgctcagcattgatcatctcacagaccacaagtcacagcgccttgcacgtctagttctgggatgcatcaccatggcatatgtgtggggcaaaggtcatggagatgtccgtaaggtcttgccaagaaatattgctgttccttactgccaactctccaagaaactggaactgcctcctattttggtttatgcagactgtgtcttggcaaactggaagaaaaaggatcctaataagcccctgacttatgagaacatggacgttttgttctcatttcgtgatggagactgcagtaaaggattcttcctggtctctctattggtggaaatagcagctgcttctgcaatcaaagtaattcctactgtattcaaggcaatgcaaatgcaagaacgggacactttgctaaaggcgctgttggaaatagcttcttgcttggagaaagcccttcaagtgtttcaccaaatccacgatcatgtgaacccaaaagcatttttcagtgttcttcgcatatatttgtctggctggaaaggcaacccccagctatcagacggtctggtgtatgaagggttctgggaagacccaaaggagtttgcagggggcagtgcaggccaaagcagcgtctttcagtgctttgacgtcctgctgggcatccagcagactgctggtggaggacatgctgctcagttcctccaggacatgagaagatatatgccaccagctcacaggaacttcctgtgctcattagagtcaaatccctcagtccgtgagtttgtcctttcaaaaggtgatgctggcctgcgggaagcttatgacgcctgtgtgaaagctctggtctccctgaggagctaccatctgcaaatcgtgactaagtacatcctgattcctgcaagccagcagccaaaggagaataagacctctgaagacccttcaaaactggaagccaaaggaactggaggcactgatttaatgaatttcctgaagactgtaagaagtacaactgagaaatcccttttgaaggaaggttaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatggctccggagagggcaggggaagtcttctaacatgcggggacgtggaggaaaatcccggccccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 14 - protein, 709 aa, Artificial Sequence
  Synthetic - PIDO fusion protein comprising human PD-L1
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEETSGGGGSGGGGSGGGGSMAHAMENSWTISKEYHIDEEVGFALPNPQENLPDFYNDWMFIAKHLPDLIESGQLRERVEKLNMLSIDHLTDHKSQRLARLVLGCITMAYVWGKGHGDVRKVLPRNIAVPYCQLSKKLELPPILVYADCVLANWKKKDPNKPLTYENMDVLFSFRDGDCSKGFFLVSLLVEIAAASAIKVIPTVFKAMQMQERDTLLKALLEIASCLEKALQVFHQIHDHVNPKAFFSVLRIYLSGWKGNPQLSDGLVYEGFWEDPKEFAGGSAGQSSVFQCFDVLLGIQQTAGGGHAAQFLQDMRRYMPPAHRNFLCSLESNPSVREFVLSKGDAGLREAYDACVKALVSLRSYHLQIVTKYILIPASQQPKENKTSEDPSKLEAKGTGGTDLMNFLKTVRSTTEKSLLKEG

SEQ ID NO: 15 - DNA, 2130 bp, Artificial Sequence
  Synthetic - DNA encoding PIDO fusion protein comprising human PD-L1
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtctggtggcggaggctcgggcggaggtgggtcgggtggcggcggatcaatggcacacgctatggaaaactcctggacaatcagtaaagagtaccatattgatgaagaagtgggctttgctctgccaaatccacaggaaaatctacctgatttttataatgactggatgttcattgctaaacatctgcctgatctcatagagtctggccagcttcgagaaagagttgagaagttaaacatgctcagcattgatcatctcacagaccacaagtcacagcgccttgcacgtctagttctgggatgcatcaccatggcatatgtgtggggcaaaggtcatggagatgtccgtaaggtcttgccaagaaatattgctgttccttactgccaactctccaagaaactggaactgcctcctattttggtttatgcagactgtgtcttggcaaactggaagaaaaaggatcctaataagcccctgacttatgagaacatggacgttttgttctcatttcgtgatggagactgcagtaaaggattcttcctggtctctctattggtggaaatagcagctgcttctgcaatcaaagtaattcctactgtattcaaggcaatgcaaatgcaagaacgggacactttgctaaaggcgctgttggaaatagcttcttgcttggagaaagcccttcaagtgtttcaccaaatccacgatcatgtgaacccaaaagcatttttcagtgttcttcgcatatatttgtctggctggaaaggcaacccccagctatcagacggtctggtgtatgaagggttctgggaagacccaaaggagtttgcagggggcagtgcaggccaaagcagcgtctttcagtgctttgacgtcctgctgggcatccagcagactgctggtggaggacatgctgctcagttcctccaggacatgagaagatatatgccaccagctcacaggaacttcctgtgctcattagagtcaaatccctcagtccgtgagtttgtcctttcaaaaggtgatgctggcctgcgggaagcttatgacgcctgtgtgaaagctctggtctccctgaggagctaccatctgcaaatcgtgactaagtacatcctgattcctgcaagccagcagccaaaggagaataagacctctgaagacccttcaaaactggaagccaaaggaactggaggcactgatttaatgaatttcctgaagactgtaagaagtacaactgagaaatcccttttgaaggaaggttaa

SEQ ID NO: 16 - DNA, 10356 bp, Artificial Sequence
  Synthetic - Lentiviral vector encoding PIDO fusion protein comprising human PD-L1
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggcaagtttgtacaaaaaagcaggctgccaccatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtctggtggcggaggctcgggcggaggtgggtcgggtggcggcggatcaatggcacacgctatggaaaactcctggacaatcagtaaagagtaccatattgatgaagaagtgggctttgctctgccaaatccacaggaaaatctacctgatttttataatgactggatgttcattgctaaacatctgcctgatctcatagagtctggccagcttcgagaaagagttgagaagttaaacatgctcagcattgatcatctcacagaccacaagtcacagcgccttgcacgtctagttctgggatgcatcaccatggcatatgtgtggggcaaaggtcatggagatgtccgtaaggtcttgccaagaaatattgctgttccttactgccaactctccaagaaactggaactgcctcctattttggtttatgcagactgtgtcttggcaaactggaagaaaaaggatcctaataagcccctgacttatgagaacatggacgttttgttctcatttcgtgatggagactgcagtaaaggattcttcctggtctctctattggtggaaatagcagctgcttctgcaatcaaagtaattcctactgtattcaaggcaatgcaaatgcaagaacgggacactttgctaaaggcgctgttggaaatagcttcttgcttggagaaagcccttcaagtgtttcaccaaatccacgatcatgtgaacccaaaagcatttttcagtgttcttcgcatatatttgtctggctggaaaggcaacccccagctatcagacggtctggtgtatgaagggttctgggaagacccaaaggagtttgcagggggcagtgcaggccaaagcagcgtctttcagtgctttgacgtcctgctgggcatccagcagactgctggtggaggacatgctgctcagttcctccaggacatgagaagatatatgccaccagctcacaggaacttcctgtgctcattagagtcaaatccctcagtccgtgagtttgtcctttcaaaaggtgatgctggcctgcgggaagcttatgacgcctgtgtgaaagctctggtctccctgaggagctaccatctgcaaatcgtgactaagtacatcctgattcctgcaagccagcagccaaaggagaataagacctctgaagacccttcaaaactggaagccaaaggaactggaggcactgatttaatgaatttcctgaagactgtaagaagtacaactgagaaatcccttttgaaggaaggttaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggctccggagagggcaggggaagtcttctaacatgcggggacgtggaggaaaatcccggccccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 17 - protein, 708 aa, Artificial Sequence
  Synthetic - PIDO fusion protein comprising canine PD-L1
MRMFSVFTFMAYCHLLKAFTITVSKDLYVVEYGGNVTMECKFPVEKQLNLFALIVYWEMEDKKIIQFVNGKEDLKVQHSSYSQRAQLLKDQLFLGKAALQITDVRLQDAGVYCCLIGYGGADYKRITLKVHAPYRNISQRISVDPVTSEHELMCQAEGYPEAEVIWTSSDHRVLSGKTTITNSNREEKLFNVTSTLNINATANEIFYCTFQRSGPEENNTAELVIPERLPVPASERTHFMILGPFLLLLGVVLAVTFCLKKHGRMMDVEKCCTRDRNSKKRNDIQFEETSGGGGSGGGGSGGGGSMAHAMENSWTISKEYHIDEEVGFALPNPQENLPDFYNDWMFIAKHLPDLIESGQLRERVEKLNMLSIDHLTDHKSQRLARLVLGCITMAYVWGKGHGDVRKVLPRNIAVPYCQLSKKLELPPILVYADCVLANWKKKDPNKPLTYENMDVLFSFRDGDCSKGFFLVSLLVEIAAASAIKVIPTVFKAMQMQERDTLLKALLEIASCLEKALQVFHQIHDHVNPKAFFSVLRIYLSGWKGNPQLSDGLVYEGFWEDPKEFAGGSAGQSSVFQCFDVLLGIQQTAGGGHAAQFLQDMRRYMPPAHRNFLCSLESNPSVREFVLSKGDAGLREAYDACVKALVSLRSYHLQIVTKYILIPASQQPKENKTSEDPSKLEAKGTGGTDLMNFLKTVRSTTEKSLLKEG

SEQ ID NO: 18 - DNA, 2127 bp, Artificial Sequence
  Synthetic - DNA encoding PIDO fusion protein comprising canine PD-L1
atgagaatgtttagtgtctttacattcatggcctactgccatttgctaaaagcatttacgatcacagtttctaaggacctgtatgtggtagagtatggtggcaatgtgacaatggaatgcaaattcccggtggaaaaacagttaaacttgtttgcactaatcgtctactgggaaatggaggataaaaaaattatacaatttgtgaatggaaaggaagacctgaaagttcagcacagcagctacagccagagggctcagctattgaaggaccagctcttcttggggaaggctgcgcttcagatcacagatgtgagattgcaggatgcaggggtttactgctgcttgatcggctatggcggtgctgactacaagcggattactttgaaagttcatgccccgtaccgcaacatcagccaaagaatttctgtggatcctgtcacctctgaacatgaactaatgtgtcaggctgagggttaccctgaggctgaagtcatctggacaagcagtgaccaccgagtcctgagtggcaaaaccaccatcactaattccaatagggaagagaagcttttcaatgtgaccagcacgctgaacatcaatgcaacagctaatgagattttctactgcacttttcaaagatcaggtcctgaggaaaacaatactgccgagttggtcatcccagaacgactgcccgttccagcaagtgagaggactcatttcatgattctgggacctttcctgttgcttcttggtgtagtcctggcagtcactttctgtctaaaaaaacatgggagaatgatggatgtggaaaaatgttgcacccgagataggaactcaaagaaacgaaatgatatacaatttgaagagacatctggtggcggaggctcgggcggaggtgggtcgggtggcggcggatcaatggcacacgctatggaaaactcctggacaatcagtaaagagtaccatattgatgaagaagtgggctttgctctgccaaatccacaggaaaatctacctgatttttataatgactggatgttcattgctaaacatctgcctgatctcatagagtctggccagcttcgagaaagagttgagaagttaaacatgctcagcattgatcatctcacagaccacaagtcacagcgccttgcacgtctagttctgggatgcatcaccatggcatatgtgtggggcaaaggtcatggagatgtccgtaaggtcttgccaagaaatattgctgttccttactgccaactctccaagaaactggaactgcctcctattttggtttatgcagactgtgtcttggcaaactggaagaaaaaggatcctaataagcccctgacttatgagaacatggacgttttgttctcatttcgtgatggagactgcagtaaaggattcttcctggtctctctattggtggaaatagcagctgcttctgcaatcaaagtaattcctactgtattcaaggcaatgcaaatgcaagaacgggacactttgctaaaggcgctgttggaaatagcttcttgcttggagaaagcccttcaagtgtttcaccaaatccacgatcatgtgaacccaaaagcatttttcagtgttcttcgcatatatttgtctggctggaaaggcaacccccagctatcagacggtctggtgtatgaagggttctgggaagacccaaaggagtttgcagggggcagtgcaggccaaagcagcgtctttcagtgctttgacgtcctgctgggcatccagcagactgctggtggaggacatgctgctcagttcctccaggacatgagaagatatatgccaccagctcacaggaacttcctgtgctcattagagtcaaatccctcagtccgtgagtttgtcctttcaaaaggtgatgctggcctgcgggaagcttatgacgcctgtgtgaaagctctggtctccctgaggagctaccatctgcaaatcgtgactaagtacatcctgattcctgcaagccagcagccaaaggagaataagacctctgaagacccttcaaaactggaagccaaaggaactggaggcactgatttaatgaatttcctgaagactgtaagaagtacaactgagaaatcccttttgaaggaaggttaa

SEQ ID NO: 19 - DNA, 10353 bp, Artificial Sequence
  Synthetic - Lentiviral vector encoding PIDO fusion protein comprising canine PD-L1
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggcaagtttgtacaaaaaagcaggctgccaccatgagaatgtttagtgtctttacattcatggcctactgccatttgctaaaagcatttacgatcacagtttctaaggacctgtatgtggtagagtatggtggcaatgtgacaatggaatgcaaattcccggtggaaaaacagttaaacttgtttgcactaatcgtctactgggaaatggaggataaaaaaattatacaatttgtgaatggaaaggaagacctgaaagttcagcacagcagctacagccagagggctcagctattgaaggaccagctcttcttggggaaggctgcgcttcagatcacagatgtgagattgcaggatgcaggggtttactgctgcttgatcggctatggcggtgctgactacaagcggattactttgaaagttcatgccccgtaccgcaacatcagccaaagaatttctgtggatcctgtcacctctgaacatgaactaatgtgtcaggctgagggttaccctgaggctgaagtcatctggacaagcagtgaccaccgagtcctgagtggcaaaaccaccatcactaattccaatagggaagagaagcttttcaatgtgaccagcacgctgaacatcaatgcaacagctaatgagattttctactgcacttttcaaagatcaggtcctgaggaaaacaatactgccgagttggtcatcccagaacgactgcccgttccagcaagtgagaggactcatttcatgattctgggacctttcctgttgcttcttggtgtagtcctggcagtcactttctgtctaaaaaaacatgggagaatgatggatgtggaaaaatgttgcacccgagataggaactcaaagaaacgaaatgatatacaatttgaagagacatctggtggcggaggctcgggcggaggtgggtcgggtggcggcggatcaatggcacacgctatggaaaactcctggacaatcagtaaagagtaccatattgatgaagaagtgggctttgctctgccaaatccacaggaaaatctacctgatttttataatgactggatgttcattgctaaacatctgcctgatctcatagagtctggccagcttcgagaaagagttgagaagttaaacatgctcagcattgatcatctcacagaccacaagtcacagcgccttgcacgtctagttctgggatgcatcaccatggcatatgtgtggggcaaaggtcatggagatgtccgtaaggtcttgccaagaaatattgctgttccttactgccaactctccaagaaactggaactgcctcctattttggtttatgcagactgtgtcttggcaaactggaagaaaaaggatcctaataagcccctgacttatgagaacatggacgttttgttctcatttcgtgatggagactgcagtaaaggattcttcctggtctctctattggtggaaatagcagctgcttctgcaatcaaagtaattcctactgtattcaaggcaatgcaaatgcaagaacgggacactttgctaaaggcgctgttggaaatagcttcttgcttggagaaagcccttcaagtgtttcaccaaatccacgatcatgtgaacccaaaagcatttttcagtgttcttcgcatatatttgtctggctggaaaggcaacccccagctatcagacggtctggtgtatgaagggttctgggaagacccaaaggagtttgcagggggcagtgcaggccaaagcagcgtctttcagtgctttgacgtcctgctgggcatccagcagactgctggtggaggacatgctgctcagttcctccaggacatgagaagatatatgccaccagctcacaggaacttcctgtgctcattagagtcaaatccctcagtccgtgagtttgtcctttcaaaaggtgatgctggcctgcgggaagcttatgacgcctgtgtgaaagctctggtctccctgaggagctaccatctgcaaatcgtgactaagtacatcctgattcctgcaagccagcagccaaaggagaataagacctctgaagacccttcaaaactggaagccaaaggaactggaggcactgatttaatgaatttcctgaagactgtaagaagtacaactgagaaatcccttttgaaggaaggttaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggctccggagagggcaggggaagtcttctaacatgcggggacgtggaggaaaatcccggccccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 20 - protein, 714 aa, Artificial Sequence
  Synthetic - PIDO fusion protein comprising feline PD-L1
MRIFSVFAFMAYCHLLKAFTITVSKDLYVVEYGSNVTMECRFPVEEQLDLVSLIVYWEMEDKKIIQFVQGKEDLKVQHRSYSQRAQLLKDQLFLGKAALQITNVTLEDAGVYCCLIGYGGADYKRITLKVHAPYRKINQRISVDPVTSEHELMCQAEGYPTAEVIWTNSAHQVLNGKTIISVSNMETKLFNVTSTLRINTTANEIFYCTFLQRSSPEGNSTAELVIPEPFLVPANERTHFMILGAILLFLVVVPAVTFCLKKRDVRTMDVEKCDTADMNSKKQNDLQFEETSGGGGSGGGGSGGGGSMSHNRILPTKNSWKNLKEYHIDEKVGFVLPTPQEELPYPYDQWISIAKNLPELIDKNELRKEVEKLKMVSIDDLKDHKSQRLAHLVLGYITMAYVWNQGGEDVCEVLPKNIAVPYCELSKKLDLPPILVYADCVLANWKKKDPNGPMTYENMDILFSFPGGDCGKGFFLVSLLVEIAAASAIKVIPDLLNAVKCEDHNTLQRALRYIASCLKQAKEEFKQIHEYVDPNTFFNVLRIYLSGWKGNPLLPEGLKYEGVWETPKKFAGGSAAQSSVFQCFDVLLGIQQSSGEESSFKFLQEMREYMPLAHRKFLQSLESAPSVREFVISKGDAKLRADYNECVKAMADLRKYHLKIVAKYIVIPSKNKHKTNSTSEETSEPENKGTGGTSVMTFLKSVRNTTEKFLLNVD

SEQ ID NO: 21 - DNA, 2145 bp, Artificial Sequence
  Synthetic - DNA encoding PIDO fusion protein comprising feline PD-L1
atgaggatatttagtgtctttgcattcatggcctactgtcatttgctgaaagcgtttacgatcacagtgtccaaggacctgtatgtggtagagtacggcagcaatgtgacaatggagtgcagattccccgtagaagaacaattagacctggtttcactgatcgtctactgggaaatggaggataagaaaatcattcagtttgtgcaagggaaggaagacctgaaagttcagcacagaagctacagtcagagggcccagctgttgaaggaccagctcttcctggggaaggccgcgcttcagatcacaaacgtgaccctggaggatgccggggtttactgctgcttgattggctatggcggtgctgactataagcggattactttgaaagttcatgccccatatcgaaaaatcaaccaaagaatttctgtggatcctgtcacctctgaacatgaactaatgtgtcaggctgagggttacccaaccgctgaagtcatctggacaaacagtgcccatcaagtcctgaatggcaaaaccatcatctctgtttccaatatggagacaaagcttttcaatgtgaccagcacgctgagaatcaacacaacggctaacgagattttctactgcacttttcttcaaagatcaagtcccgagggaaacagtactgctgagttggtcatcccagaaccatttctggttccagcaaatgagaggactcacttcatgattctaggagccatcctgttgtttcttgtcgtggtcccggctgtcactttctgtctgaagaaacgagatgtacgaacgatggatgtggaaaaatgtgacaccgcagatatgaactcaaagaagcaaaatgatctacaatttgaggagacgtctggtggcggaggctcgggcggaggtgggtcgggtggcggcggatcaatgtcacacaatagaatattgcctacaaaaaattcttggaaaaatttgaaagaataccatatagatgaaaaggtgggcttcgttctgccaactccacaggaggaactaccttacccttatgatcaatggatttccattgctaaaaacctgcctgaacttattgacaaaaatgaactacgtaaagaagttgagaagttaaaaatggtcagtattgatgacctcaaagaccacaagtcacagcgccttgcacatctggtcctggggtatatcaccatggcgtatgtgtggaaccaaggcggtgaagatgtttgtgaggtcttgccaaagaacattgctgttccttactgtgaactgtctaagaagctggatctgcctcctattctggtttatgcagattgtgtcttggcaaactggaagaaaaaggatcccaatgggcccatgacttatgagaacatggacattctgttttcgtttcctggtggagactgcggtaaaggatttttcctggtttctctgttggtggaaatagcagcggcttctgcaatcaaagtaattcctgatttattgaatgcagtaaaatgtgaggaccacaatactctgcaaagggcgctgcgttacatagcttcttgtctgaaacaagccaaggaagagtttaaacaaattcatgaatatgtggacccaaacacatttttcaatgttcttcgcatatacttgtctggctggaaaggcaaccccctgctgccagagggtctgaagtatgaaggtgtctgggaaaccccaaagaagtttgcagggggtagtgcagcccaaagcagtgtcttccagtgctttgatgttctgctgggcatccagcagagttctggtgaagaatcttctttcaaattccttcaggaaatgagagaatatatgccattagctcaccggaaatttctccagtcattagagtcagccccctcagtccgtgagtttgttatttcaaaaggtgatgctaaactgagagcagattataatgaatgtgtgaaagctatggctgacctgagaaaataccatttgaaaatagtagccaagtacatcgtgattccttcaaagaacaagcacaagacaaactcaacatctgaagaaacatcagaaccagaaaataaaggaactggaggcactagtgtcatgactttcctgaagagtgttagaaatacaactgagaaattcctgctgaacgttgactaa

SEQ ID NO: 22 - DNA, 10937 bp, Artificial Sequence
  Synthetic - Lentiviral vector encoding PIDO fusion protein comprising feline PD-L1
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcaagtttgtacaaaaaagcaggctgccaccatgaggatatttagtgtctttgcattcatggcctactgtcatttgctgaaagcgtttacgatcacagtgtccaaggacctgtatgtggtagagtacggcagcaatgtgacaatggagtgcagattccccgtagaagaacaattagacctggtttcactgatcgtctactgggaaatggaggataagaaaatcattcagtttgtgcaagggaaggaagacctgaaagttcagcacagaagctacagtcagagggcccagctgttgaaggaccagctcttcctggggaaggccgcgcttcagatcacaaacgtgaccctggaggatgccggggtttactgctgcttgattggctatggcggtgctgactataagcggattactttgaaagttcatgccccatatcgaaaaatcaaccaaagaatttctgtggatcctgtcacctctgaacatgaactaatgtgtcaggctgagggttacccaaccgctgaagtcatctggacaaacagtgcccatcaagtcctgaatggcaaaaccatcatctctgtttccaatatggagacaaagcttttcaatgtgaccagcacgctgagaatcaacacaacggctaacgagattttctactgcacttttcttcaaagatcaagtcccgagggaaacagtactgctgagttggtcatcccagaaccatttctggttccagcaaatgagaggactcacttcatgattctaggagccatcctgttgtttcttgtcgtggtcccggctgtcactttctgtctgaagaaacgagatgtacgaacgatggatgtggaaaaatgtgacaccgcagatatgaactcaaagaagcaaaatgatctacaatttgaggagacgtctggtggcggaggctcgggcggaggtgggtcgggtggcggcggatcaatgtcacacaatagaatattgcctacaaaaaattcttggaaaaatttgaaagaataccatatagatgaaaaggtgggcttcgttctgccaactccacaggaggaactaccttacccttatgatcaatggatttccattgctaaaaacctgcctgaacttattgacaaaaatgaactacgtaaagaagttgagaagttaaaaatggtcagtattgatgacctcaaagaccacaagtcacagcgccttgcacatctggtcctggggtatatcaccatggcgtatgtgtggaaccaaggcggtgaagatgtttgtgaggtcttgccaaagaacattgctgttccttactgtgaactgtctaagaagctggatctgcctcctattctggtttatgcagattgtgtcttggcaaactggaagaaaaaggatcccaatgggcccatgacttatgagaacatggacattctgttttcgtttcctggtggagactgcggtaaaggatttttcctggtttctctgttggtggaaatagcagcggcttctgcaatcaaagtaattcctgatttattgaatgcagtaaaatgtgaggaccacaatactctgcaaagggcgctgcgttacatagcttcttgtctgaaacaagccaaggaagagtttaaacaaattcatgaatatgtggacccaaacacatttttcaatgttcttcgcatatacttgtctggctggaaaggcaaccccctgctgccagagggtctgaagtatgaaggtgtctgggaaaccccaaagaagtttgcagggggtagtgcagcccaaagcagtgtcttccagtgctttgatgttctgctgggcatccagcagagttctggtgaagaatcttctttcaaattccttcaggaaatgagagaatatatgccattagctcaccggaaatttctccagtcattagagtcagccccctcagtccgtgagtttgttatttcaaaaggtgatgctaaactgagagcagattataatgaatgtgtgaaagctatggctgacctgagaaaataccatttgaaaatagtagccaagtacatcgtgattccttcaaagaacaagcacaagacaaactcaacatctgaagaaacatcagaaccagaaaataaaggaactggaggcactagtgtcatgactttcctgaagagtgttagaaatacaactgagaaattcctgctgaacgttgactaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggctccggagagggcaggggaagtcttctaacatgcggggacgtggaggaaaatcccggccccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 23 - protein, 289 aa, Canis lupus
  MISC_FEATURE (1)..(289): Full-length canine PD-L1 protein
  MISC_FEATURE (121)..(125): ADYKR sequence required for PD-1 binding domain
MRMFSVFTFMAYCHLLKAFTITVSKDLYVVEYGGNVTMECKFPVEKQLNLFALIVYWEMEDKKIIQFVNGKEDLKVQHSSYSQRAQLLKDQLFLGKAALQITDVRLQDAGVYCCLIGYGGADYKRITLKVHAPYRNISQRISVDPVTSEHELMCQAEGYPEAEVIWTSSDHRVLSGKTTITNSNREEKLFNVTSTLNINATANEIFYCTFQRSGPEENNTAELVIPERLPVPASERTHFMILGPFLLLLGVVLAVTFCLKKHGRMMDVEKCCTRDRNSKKRNDIQFEET

SEQ ID NO: 24 - protein, 291 aa, Felis catus
  MISC_FEATURE (1)..(291): Full-length feline PD-L1 protein
  MISC_FEATURE (121)..(125): ADYKR sequence required for PD-1 binding domain
MRIFSVFAFMAYCHLLKAFTITVSKDLYVVEYGSNVTMECRFPVEEQLDLVSLIVYWEMEDKKIIQFVQGKEDLKVQHRSYSQRAQLLKDQLFLGKAALQITNVTLEDAGVYCCLIGYGGADYKRITLKVHAPYRKINQRISVDPVTSEHELMCQAEGYPTAEVIWTNSAHQVLNGKTIISVSNMETKLFNVTSTLRINTTANEIFYCTFLQRSSPEGNSTAELVIPEPFLVPANERTHFMILGAILLFLVVVPAVTFCLKKRDVRTMDVEKCDTADMNSKKQNDLQFEET